(19) United States
(12) Patent Application Publication
(10) Pub. No.: US 2024/0399090 A1
(43) Pub. Date: Dec. 5, 2024

(54) APPARATUS FOR REPURPOSING PAP THERAPY

(71) Applicants: ResMed Pty Ltd, Bella Vista (AU); ResMed Asia Pacific Limited, Bella Vista (AU)

(72) Inventors: Richard Charles NAPPER, Sydney (AU); Andrew CHAN, Sydney (AU); Stewart Joseph WAGNER, Hawkesbury (AU)

(21) Appl. No.: 18/677,775

(22) Filed: May 29, 2024

(30) Foreign Application Priority Data
May 29, 2023 (AU) 2023901671

(51) Int. Cl. A61M 16/06; A61M 16/00; A61M 16/20

(52) U.S. Cl. CPC A61M 16/0605; A61M 16/024; A61M 16/208

(57) ABSTRACT

Disclosed is an apparatus for delivery of pressurised air or breathable gas to a patient. The apparatus may comprise a flow generator configured to generate a flow of air. A patient interface may be constructed and arranged to form a seal with a region of the patient's face surrounding an entrance to the patient's airways. The patient interface may be configured to deliver the pressurised air or breathable gas to the patient's airways for respiratory therapy. An air delivery tube may be coupled between the flow generator and the patient interface to deliver the flow of air from the flow generator to the patient interface as the pressurised air or breathable gas. A complementary flow device may be configured to divert at least part of the pressurised air or breathable gas away from the patient's airway.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

### 1.2.3.2 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal-forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 1.2.3.3 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534.

Another technique is the use of one or more straps and/or stabilising harnesses. Many such harnesses suffer from being one or more of ill-fitting, bulky, uncomfortable and awkward to use.

### 1.2.3.4 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 1.2.3.4.1 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 1.2.3.5 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices (one specimen only, measured using test method specified in ISO 3744 in CPAP mode at 10 cmH2O).

| | A-weighted | |
|---|---|---|
| | sound | |
| | pressure | |
| | level | Year |
| RPT Device name | dB(A) | (approx.) |
| C-Series Tango ™ | 31.9 | 2007 |
| C-Series Tango ™ with Humidifier | 33.1 | 2007 |
| S8 Escape ™ II | 30.5 | 2005 |
| S8 Escape ™ II with H4i ™ Humidifier | 31.1 | 2005 |
| S9 AutoSet ™ | 26.5 | 2010 |
| S9 AutoSet ™ with H5i Humidifier | 28.6 | 2010 |

One known RPT device used for treating sleep disordered breathing is the S9 Sleep Therapy System, manufactured by ResMed Inc. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.6 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.7 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers

While a number of medical humidifiers are known, they can suffer from one or more shortcomings. Some medical humidifiers may provide inadequate humidification, some are difficult or inconvenient to use by patients.

### 1.2.3.8 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.9 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Inc. has developed a number of improved mask vent technologies, e.g. see International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1 m)

| | | A-weighted | A-weighted | |
|---|---|---|---|---|
| | | sound power | sound pressure | |
| | | level dB(A) | dB(A) | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed | nasal | 32 (3) | 24 (3) | 2002 |
| Mirage | | | | |
| Activa ™ | | | | |
| ResMed | nasal | 30 (3) | 22 (3) | 2008 |
| Mirage | | | | |
| Micro ™ | | | | |
| ResMed | nasal | 29 (3) | 22 (3) | 2008 |
| Mirage ™ | | | | |
| SoftGel | | | | |
| ResMed | nasal | 26 (3) | 18 (3) | 2010 |
| Mirage ™ FX | | | | |
| ResMed | nasal | 37 | 29 | 2004 |
| Mirage | pillows | | | |
| Swift ™ (*) | | | | |
| ResMed | nasal | 28 (3) | 20 (3) | 2005 |
| Mirage | pillows | | | |
| Swift ™ II | | | | |
| ResMed | nasal | 25 (3) | 17 (3) | 2008 |
| Mirage | pillows | | | |
| Swift ™ LT | | | | |
| ResMed AirFit | nasal | 21 (3) | 13 (3) | 2014 |
| P10 | pillows | | | |

(*one specimen only, measured using test method specified in ISO 3744 in CPAP mode at 10 cmH2O)

Sound pressure values of a variety of objects are listed below

| | A-weighted | |
|---|---|---|
| | sound | |
| | pressure | |
| Object | dB(A) | Notes |
| Vacuum cleaner: Nilfisk | 68 | ISO 3744 at |
| Walter Broadly Litter Hog: | | 1 m distance |
| B+ Grade | | |
| Conversational speech | 60 | 1 m distance |
| Average home | 50 | |
| Quiet library | 40 | |
| Quiet bedroom at night | 30 | |
| Background in TV studio | 20 | |

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises an apparatus for delivery of pressurised air or breathable gas to a patient. The apparatus may comprise a flow generator configured to generate a flow of air. A patient interface may be constructed and arranged to form a seal with a region of the patient's face surrounding an entrance to the patient's airways. The patient interface may be configured to deliver the pressurised air or breathable gas to the patient's airways for respiratory therapy. An air delivery tube may be coupled between the flow generator and the patient interface to deliver a first portion of the flow of air from the flow generator to the patient interface as the pressurised air or breathable gas. A complementary flow device may be configured to deliver a second portion of the flow of air as a complementary activity to the respiratory therapy.

An aspect of one form of the present technology, is that the complementary flow device may be configured to deliver the second portion of the flow of air away from the patient's airway.

An aspect of one form of the present technology, is that the complementary flow device may be configured to deliver the second portion of the flow of air outside the patient interface.

Another aspect of one form of the present technology, is that the complementary flow device may be configured to deliver the second portion of the flow of air onto the patient's skin.

Another aspect of one form of the present technology, is that the complementary flow device is configured to target a discrete location of the patient's skin. The second portion of the flow of air may stimulate a response in the patient that forms at least part of an active complementary therapy.

Another aspect of one form of the present technology, is that the complementary flow device may be configured to diffuse the second portion of the flow of air across the patient's skin. This may alter the environment around the patient.

Another aspect of one form of the present technology, is that a single stream of air is produced in the air tube from the flow generator and that the complementary flow device is configured to divert a portion of air from that single stream to form the second portion of the flow of air.

Another aspect of one form of the technology, is that the complementary flow device causes the flow of air from the flow generator to be in multiple streams, with one stream including the first portion of the flow of air and a second stream including the second portion of the flow of air. According to one form of the technology, the flow generator generates a single stream that is then separated into multiple streams. In one form, the flow generator is configured to generate separate streams by separate components within a unit, or by separate independent units.

Another aspect of one form of the present technology, is that the patient interface comprises a frame configured to conform to the shape of the patient's face. The complementary flow device may form part of the patient interface. The complementary flow device may also be arranged in the frame to direct the at least part of the flow of air away from the patient's airways.

Another aspect of one form of the present technology, is that the complementary flow device and the air delivery tube are coupled together by routing provided on the frame, or other component, of the patient interface.

Another aspect of one form of the present technology, may comprise more than one complementary flow device for delivering the second portion of the flow of air towards more than one location away from the patient's airways.

Another aspect of one form of the present technology, is that the more than one complementary flow device may be for delivering more than one active complementary therapy to the patient. The second portion of the flow of air may be configured to stimulate a response in the patient and/or passive complementary therapy where the second portion of flow of air, or part thereof, may alter the environment around the patient.

Another aspect of one form of the present technology, is that the complementary flow device comprises an array of apertures configured to divert the second portion of the flow of air away from the patient's airways.

Another aspect of one form of the present technology, is that the apparatus further comprising a flow regulator valve for regulating the second portion of the flow of air to the patient, the flow regulator configured to move between an open state for allowing airflow therethrough, and a closed state for blocking airflow therethrough, wherein the state of the flow regulator valve between the open and closed states is dependent on an orientation of the apparatus.

Another aspect of one form of the present technology is that the flow regulator valve may be arranged to move between the closed and open states under gravity.

One form of the present technology comprises a patient interface for delivery of pressurised air or breathable gas to a patient. The patient interface may be constructed and arranged to form a seal with a region of the patient's face surrounding an entrance to the patient's airways to deliver the pressurised air or breathable gas to the patient's airways for respiratory therapy. The patient interface may further comprise a complementary flow device. The complementary flow device may be configured to divert at least part of the pressurised air or breathable gas as a complementary activity to the respiratory therapy.

An aspect of one form of the present technology, is that the complementary flow device may be configured to divert the pressurised air or breathable gas away from the patient's airway.

An aspect of one form of the present technology, is that the complementary flow device may be configured to deliver the diverted pressurised air or breathable gas onto the patient's skin outside the patient interface.

Another aspect of one form of the present technology, is that the complementary flow device may be configured to target a discrete location of the patient's skin. The diverted flow of air may stimulate a response in the patient that forms at least part of an active complementary therapy.

Another aspect of one form of the present technology, is that the complementary flow device may be configured to diffuse the diverted flow of air across the patient's skin to alter the environment around the patient.

Another aspect of one form of the present technology, is that the diverted pressurised air or breathable gas is directed to one or more specific areas of the patient airways as the complementary activity.

Another aspect of one form of the present technology, is that the patient interface may comprise a frame configured to conform to the shape of the patient's face. The complementary flow device may form part of the patient interface. The complementary flow device may also be arranged in the frame to direct the at least part of the flow of air away from the patient's airways.

Another aspect of one form of the present technology, is that the complementary flow device includes routing provided on the frame of the patient interface that directs flow from the air delivery tube.

Another aspect of one form of the present technology, is that the patient interface may comprise more than one complementary flow device for diverting the flow of air towards more than one location away from the patient's airways.

Another aspect of one form of the present technology, is that the more than one complementary flow device can be for delivering more than one of active complementary therapy to the patient where the diverted airflow is configured to stimulate a response in the patient and/or passive complementary therapy where the diverted airflow may alter the environment around the patient.

Another aspect of one form of the present technology, is that the complementary flow device may comprise an array of apertures configured to divert the flow of air away from the patient's airway.

Another aspect of one form of the present technology, is that the patient interface further comprising a flow regulator valve for regulating the second portion of the flow of air to the patient, the flow regulator configured to move between an open state for allowing airflow therethrough, and a closed state for blocking airflow therethrough, wherein the state of the flow regulator valve between the open and closed states is dependent on an orientation of the patient interface.

Another aspect of one form of the present technology, is that the flow regulator valve may be arranged to move between the closed and open states under gravity.

One form of the present technology comprises a flow regulator valve for use with an apparatus for delivery of pressurised air or breathable gas to a patient for respiratory therapy, the flow regulator valve configured for regulating a flow of air to the patient as a complementary activity to the respiratory therapy, the flow regulator configured to move between an open state for allowing airflow therethrough, and a closed state for blocking airflow therethrough, wherein the state of the flow regulator valve between the open and closed states is dependent on an orientation of the apparatus.

Another aspect of one form of the present technology, is that the flow regulator valve is arranged to move between the closed and open states under gravity.

One form of the present technology comprises a system for delivery of pressurised air or breathable gas to a patient. The system can comprise a patient interface constructed and arranged to form a seal with a region of the patient's face surrounding an entrance to the patient's airways. The patient interface may be configured to deliver the pressurised air or breathable gas to the patient's airways for respiratory therapy. A flow generator may be configured to generate a flow of air. An air delivery tube may be coupled between the flow generator and the patient interface to deliver a first portion of the flow of air as the pressurised air or breathable gas to the patient interface. A complementary flow device may be configured to deliver a second portion of the flow of air away from the patient's airway.

An aspect of one form of the present technology, is that the system may further comprise a sensory monitoring and stimulation unit. The system may also comprise a controller configured with respect to the flow generator to set an operation of the complementary flow device.

Another aspect of one form of the present technology, is that the sensory monitoring and stimulation unit may be coupled with one or more sensors configured to detect physiological data of the patient. The controller may be configured to set the operation of the complementary flow device based on a signal from the one or more sensors.

Another aspect of one form of the present technology, is that the sensory monitoring and stimulation unit may be coupled with one or more sensors configured to detect data of the patient's sleep environment. The controller may be configured to set the operation of the complementary flow device based on a signal from the one or more sensors.

Another aspect of one form of the present technology, is that the complementary flow device may be configured to deliver the second portion of the flow of air outside the patient interface.

Another aspect of one form of the present technology, is that the complementary flow device may be configured to deliver the second portion of the flow of air onto the patient's skin.

Another aspect of one form of the present technology, is that the complementary flow device may be configured to target a discrete location of the patient's skin. The second portion of the flow of air may stimulate a response in the patient that forms at least part of an active complementary therapy.

Another aspect of one form of the present technology, is that the complementary flow device may be configured to diffuse the second portion of the flow of air across the patient's skin to alter the environment around the patient.

Another aspect of one form of the present technology, is that the patient interface may comprise a frame configured to conform to the shape of the patient's face. The complementary flow device may form part of the patient interface. The complementary flow device may also be arranged in the frame to direct the at least part of the flow of air away from the patient's airways.

Another aspect of one form of the present technology, is that the complementary flow device and the air delivery tube may be coupled together by routing provided on the frame of the patient interface.

Another aspect of one form of the present technology, is that the system may comprise more than one complementary flow device for delivering the second portion of the flow of air towards more than one location away from the patient's airways.

Another aspect of one form of the present technology, is that the more than one complementary flow device may be for delivering more than one of active complementary therapy to the patient. The second portion of the flow of air may be configured to stimulate a response in the patient and/or passive complementary therapy where the diverted airflow may alter the environment around the patient.

Another aspect of one form of the present technology, is that the complementary flow device may comprise an array of apertures configured to divert the flow of air away from the patient's airways.

One form of the present technology comprises a method for delivering pressurised air or breathable gas to a patient. The method may comprise arranging a patient interface to form a seal with a region of the patient's face surrounding an entrance to the patient's airways. The method may also comprise generating a flow of air, and delivering a first portion of the flow of air to the patient interface as the pressurised air or breathable gas. The method may also comprise delivering the pressurised air or breathable gas to the patient's airways via the patient interface for respiratory therapy. The method may also comprise delivering a second portion of the flow of gas as a complementary activity to the respiratory activity.

An aspect of one form of the present technology, is that the complementary flow device may be configured to deliver the second portion of the flow of air away from the patient's airways.

An aspect of one form of the present technology comprises delivering the second portion of the flow of air outside the patient interface.

Another aspect of one form of the present technology comprises directing the second portion of the flow of air onto the patient's skin.

Another aspect of one form of the present technology, is that the second portion of the flow of air is directed to one or more specific areas of the patient airways as the complementary activity.

Another aspect of one form of the present technology comprises sensing physiological data of the patient. The second portion of the flow of air may be delivered outside the patient interface and/or directed onto the patient's skin in response to the sensed physiological data.

Another aspect of one form of the present technology comprises sensing the patient's sleep environment. The second portion of the flow of air may be delivered outside the patient interface and/or directed onto the patient's skin in response to the sensed sleep environment.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

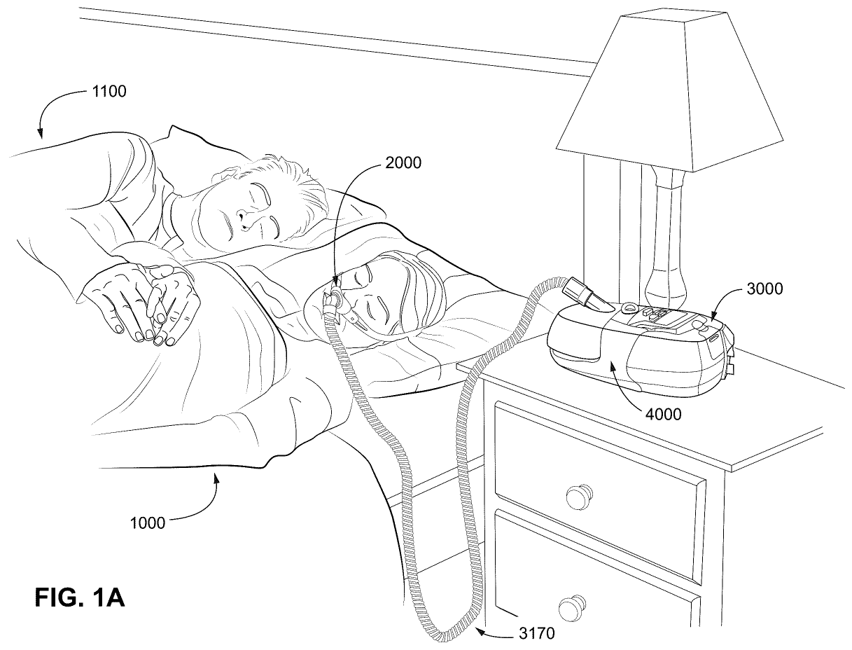

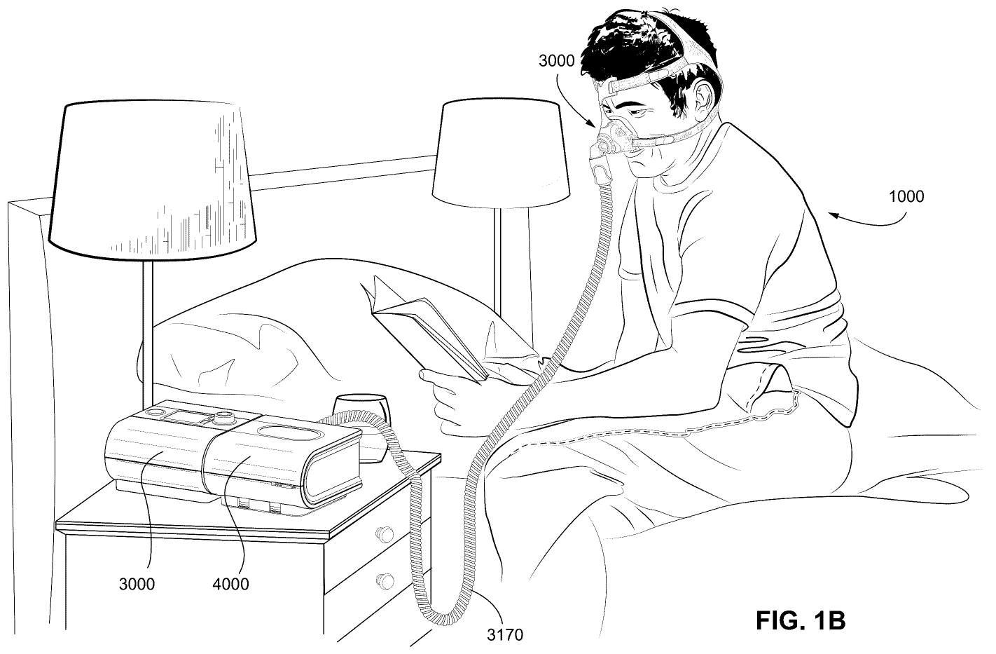

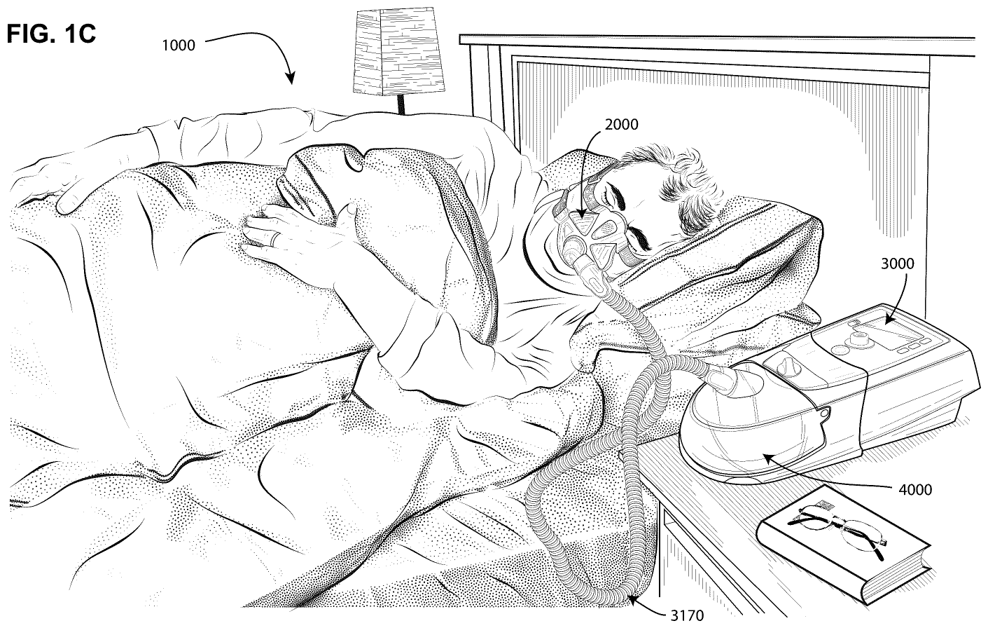

FIG. 1A shows a system including a patient 1000 wearing a patient interface 2000, in the form of nasal pillows, receiving a supply of air at positive pressure from an RPT device 3000. Air from the RPT device 3000 is humidified in a humidifier 4000, and passes along an air circuit 3170 to the patient 1000. A bed partner 1100 is also shown. The patient is sleeping in a supine sleeping position.

FIG. 1B shows a system including a patient 1000 wearing a patient interface 2000, in the form of a nasal mask, receiving a supply of air at positive pressure from an RPT device 3000. Air from the RPT device is humidified in a humidifier 4000, and passes along an air circuit 3170 to the patient 1000.

FIG. 1C shows a system including a patient 1000 wearing a patient interface 2000, in the form of a full-face mask, receiving a supply of air at positive pressure from an RPT device 3000. Air from the RPT device is humidified in a humidifier 4000, and passes along an air circuit 3170 to the patient 1000. The patient is sleeping in a side sleeping position.

### 3.2 Respiratory System and Facial Anatomy

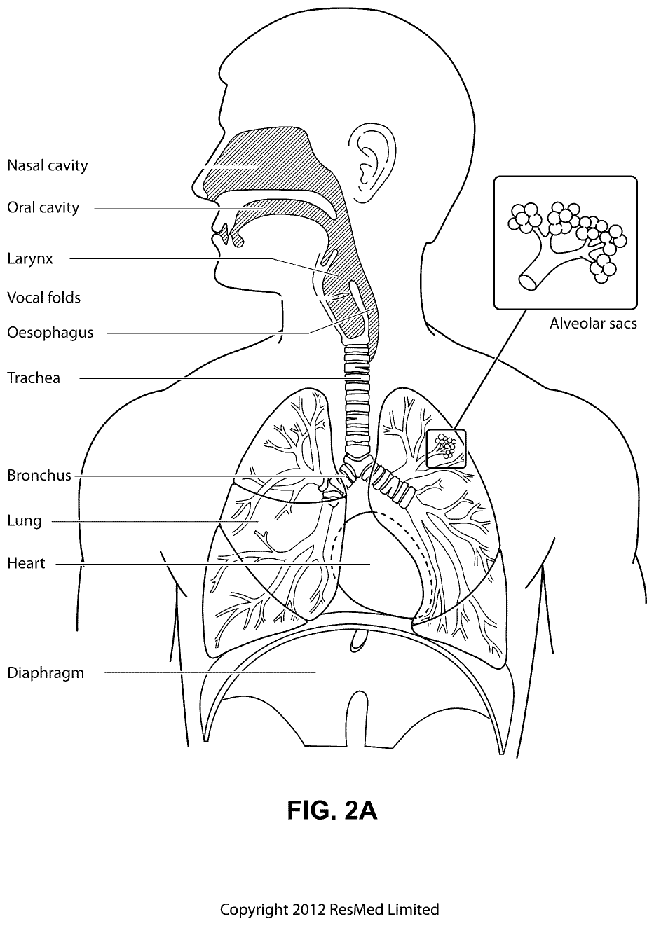

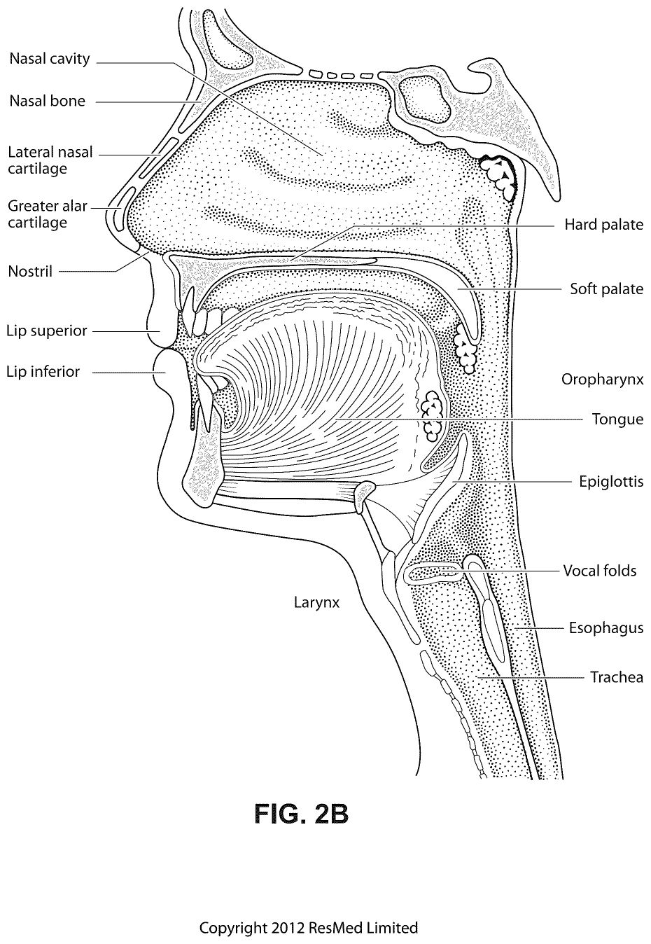

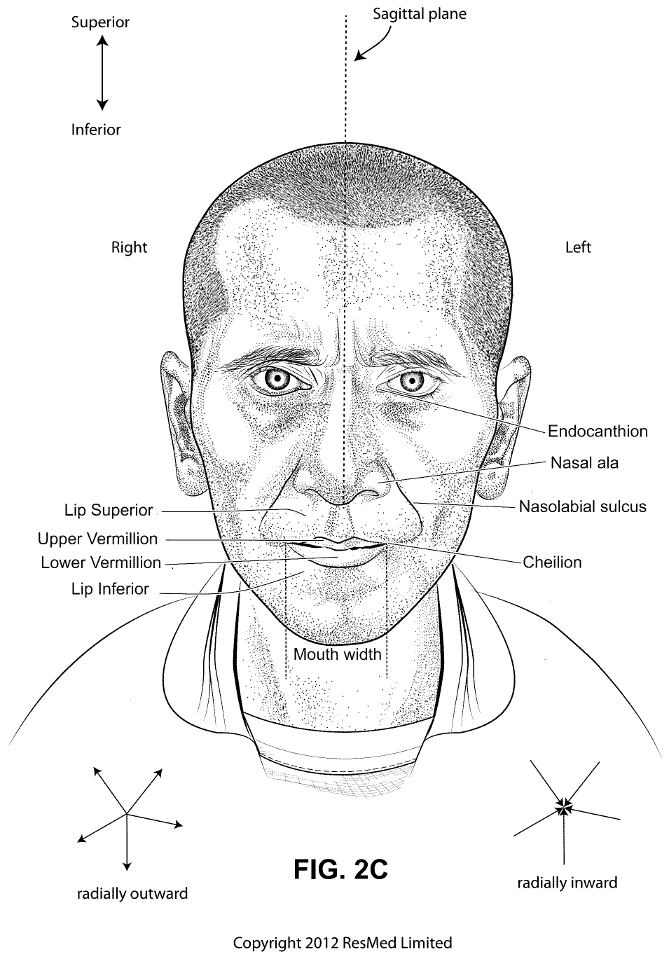

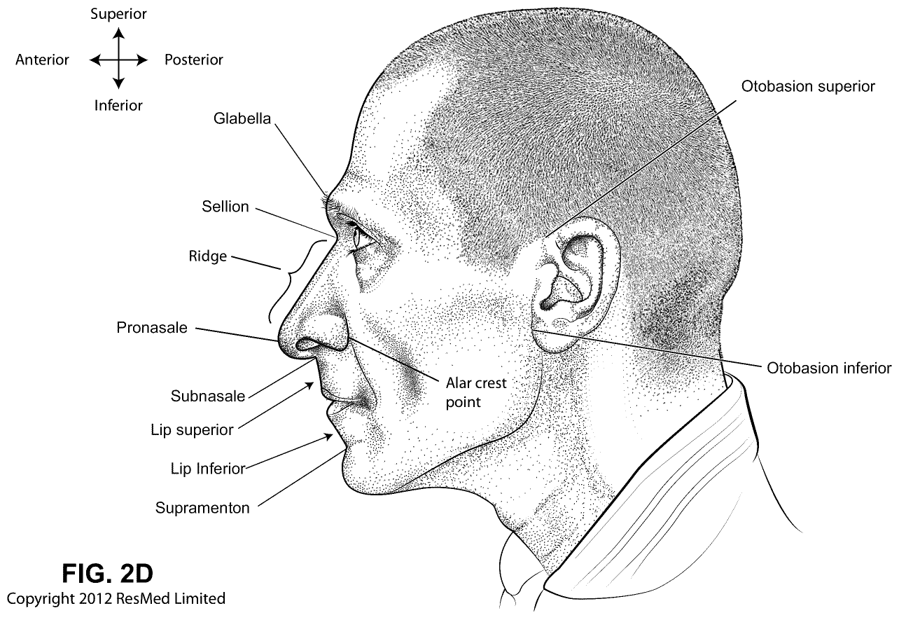

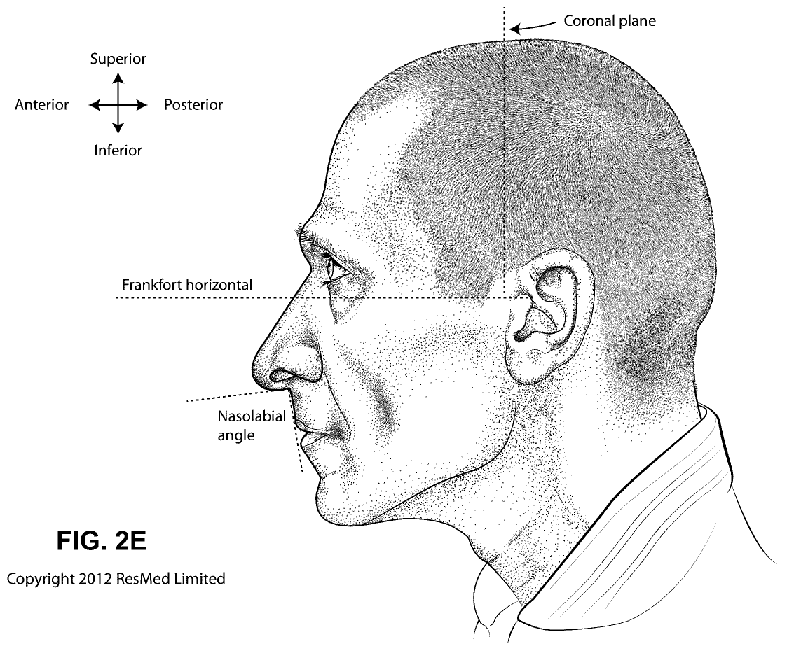

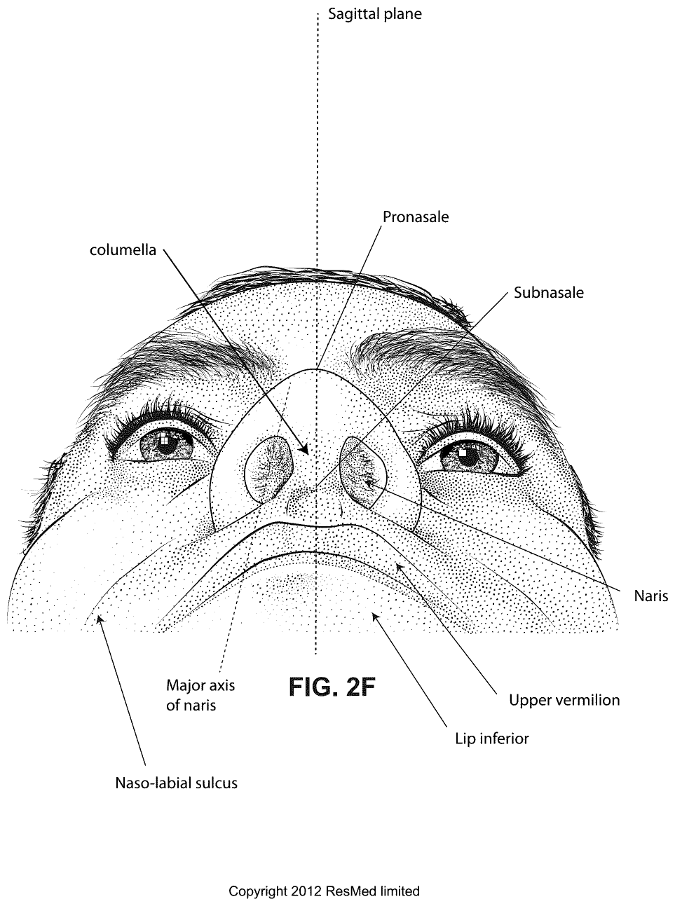

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

### 3.3 Patient Interface

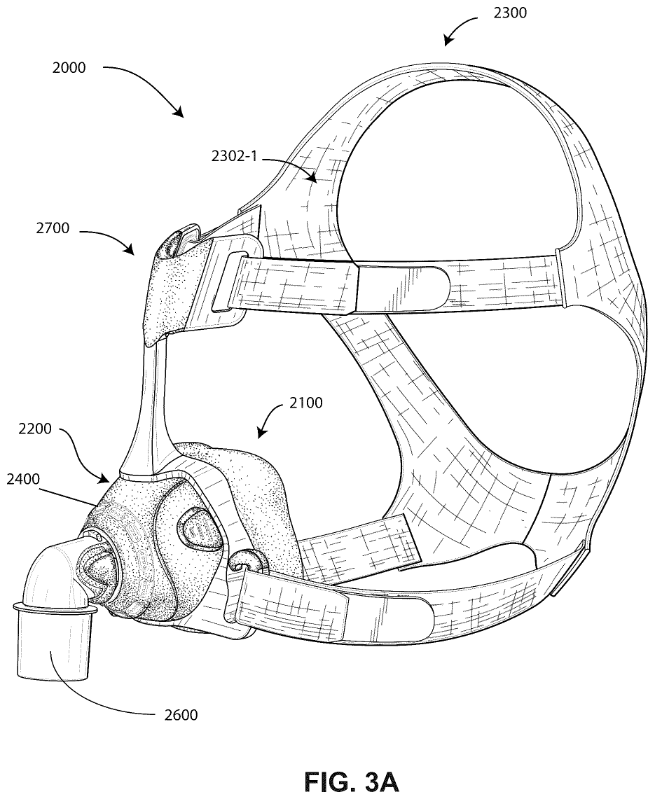

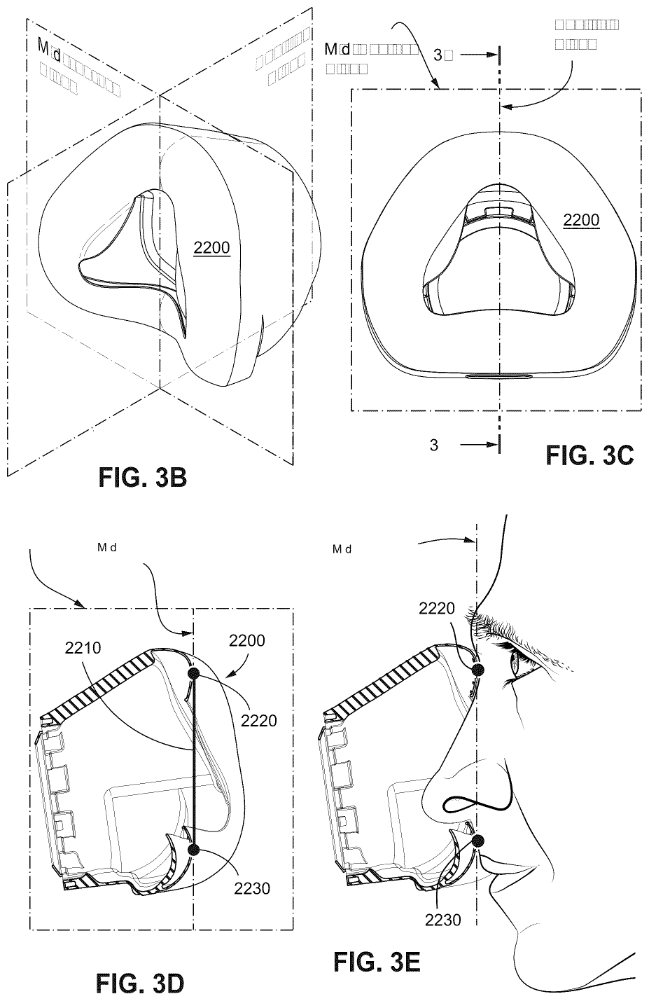

FIG. 3A shows a patient interface in the form of a nasal mask.

FIG. 3B shows a view of a plenum chamber 2200 showing a sagittal plane and a mid-contact plane.

FIG. 3C shows a view of a posterior of the plenum chamber of FIG. 3B. The direction of the view is normal to the mid-contact plane. The sagittal plane in FIG. 3D bisects the plenum chamber into left-hand and right-hand sides.

FIG. 3D shows a cross-section through the plenum chamber of FIG. 3C, the cross-section being taken at the sagittal plane shown in FIG. 3C. A ‘mid-contact’ plane is shown. The mid-contact plane is perpendicular to the sagittal plane. The orientation of the mid-contact plane corresponds to the orientation of a chord 2210 which lies on the sagittal plane and just touches the cushion of the plenum chamber at two points on the sagittal plane: a superior point 2220 and an inferior point 2230. Depending on the geometry of the cushion in this region, the mid-contact plane may be a tangent at both the superior and inferior points.

FIG. 3E shows the plenum chamber 2200 of FIG. 3B in position for use on a face. The sagittal plane of the plenum chamber 2200 generally coincides with the midsagittal plane of the face when the plenum chamber is in position for use. The mid-contact plane corresponds generally to the ‘plane of the face’ when the plenum chamber is in position for use. In FIG. 3E the plenum chamber 2200 is that of a nasal mask, and the superior point 2220 sits approximately on the sellion, while the inferior point 2230 sits on the lip superior.

FIG. 3F shows a patient interface having conduit headgear.

### 3.4 RPT Device

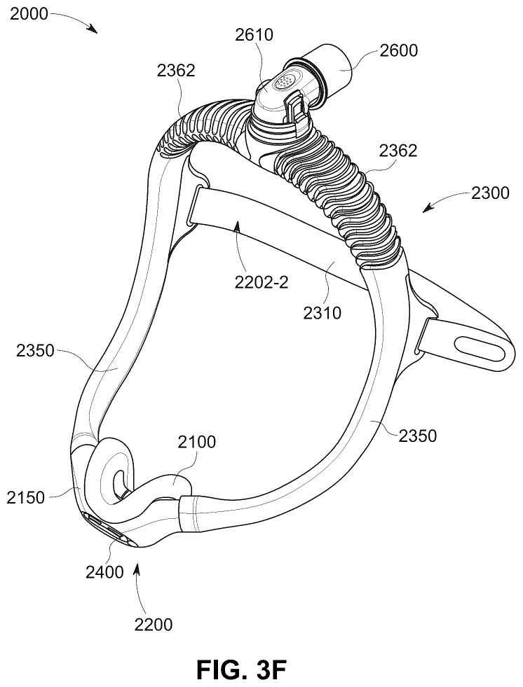

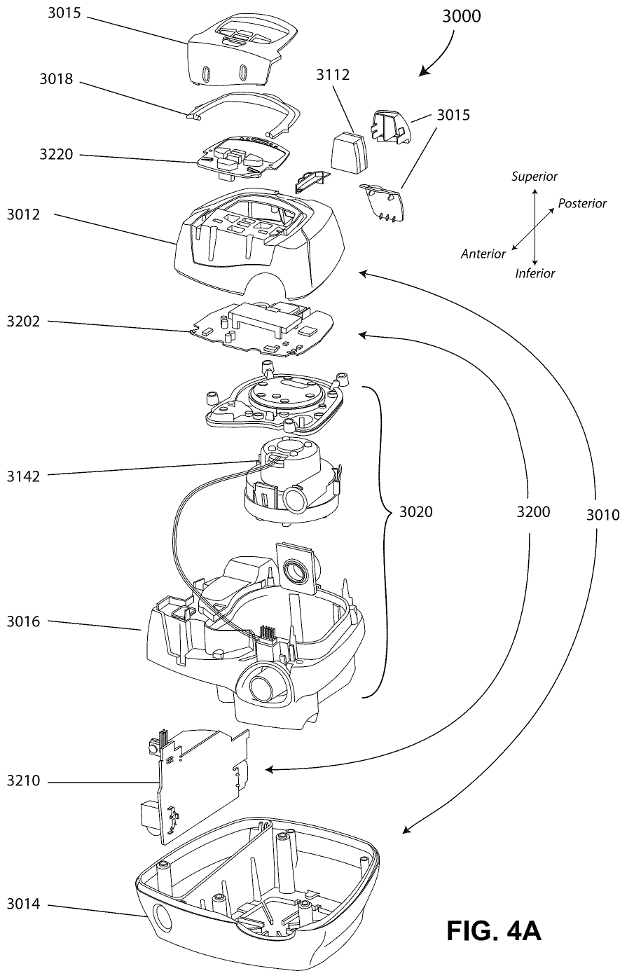

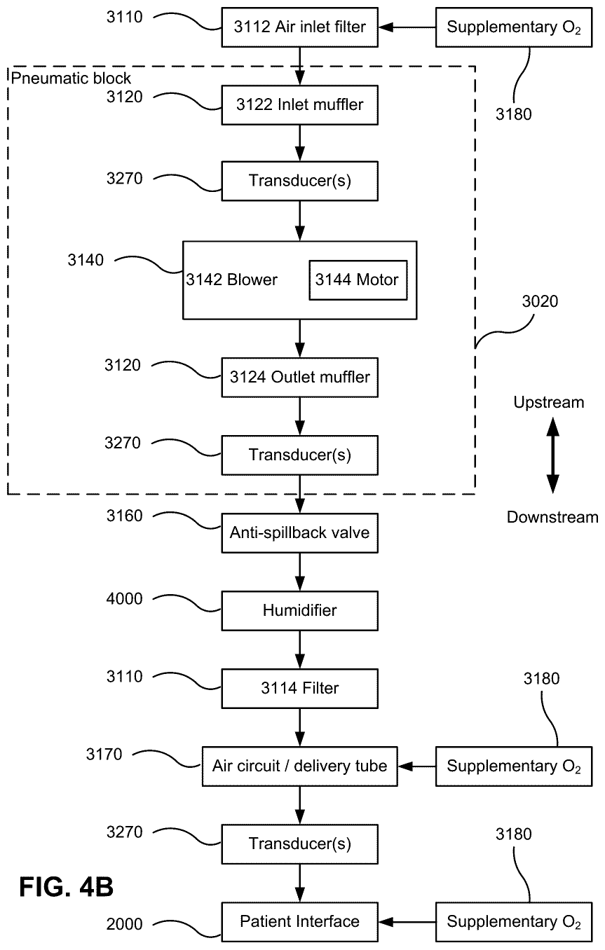

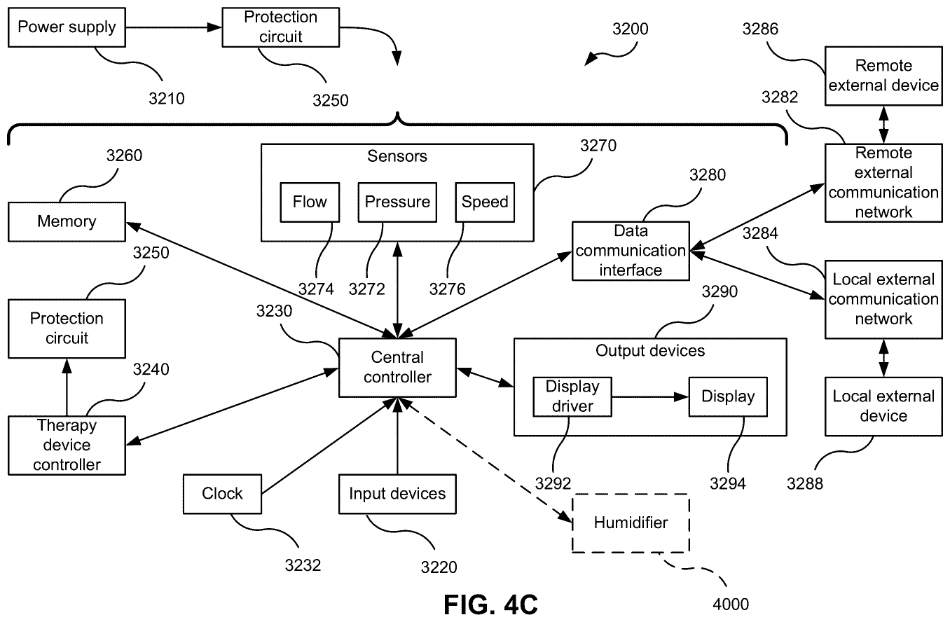

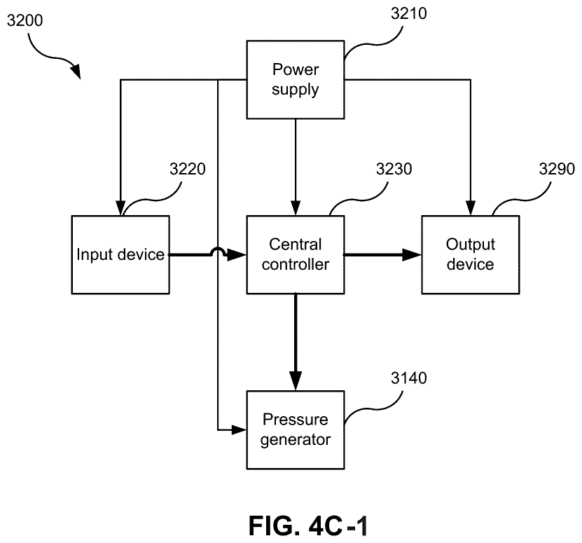

FIG. 4A shows an RPT device.

FIG. 4B is a schematic diagram of the pneumatic path of an RPT device. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device.

FIG. 4C-1 is a schematic diagram illustrating the interconnection of various electrical components of the RPT device.

### 3.5 Humidifier

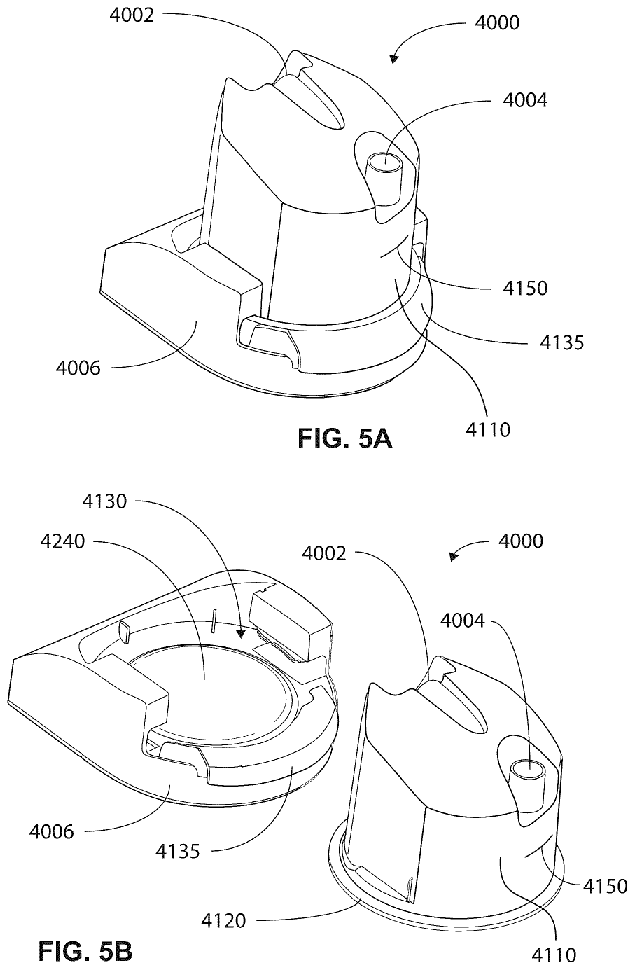

FIG. 5A shows an isometric view of a humidifier.

FIG. 5B shows an isometric view of a humidifier, showing a humidifier reservoir 4110 removed from the humidifier reservoir dock 4130.

### 3.6 Breathing Waveforms

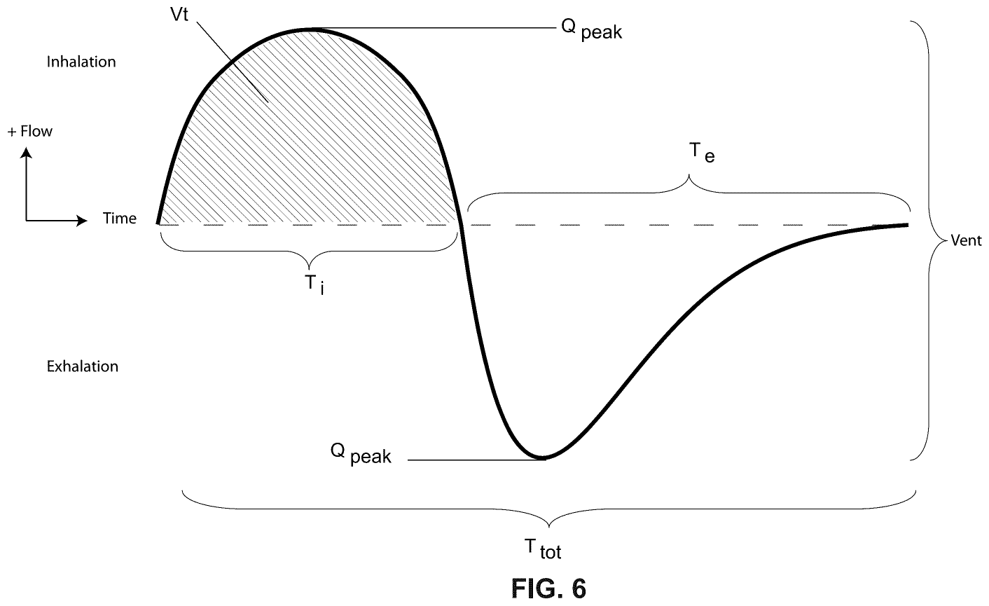

FIG. 6 shows a model typical breath waveform of a person while sleeping.

### 3.7 Complementary Therapy

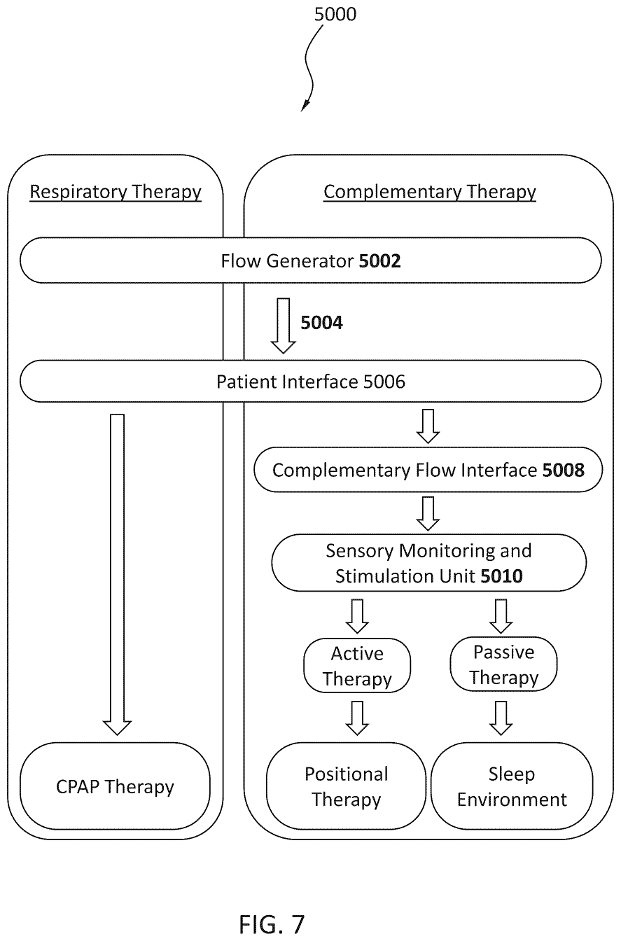

FIG. 7 is a flowchart illustrating an apparatus for providing both respiratory pressure therapies and complementary therapies.

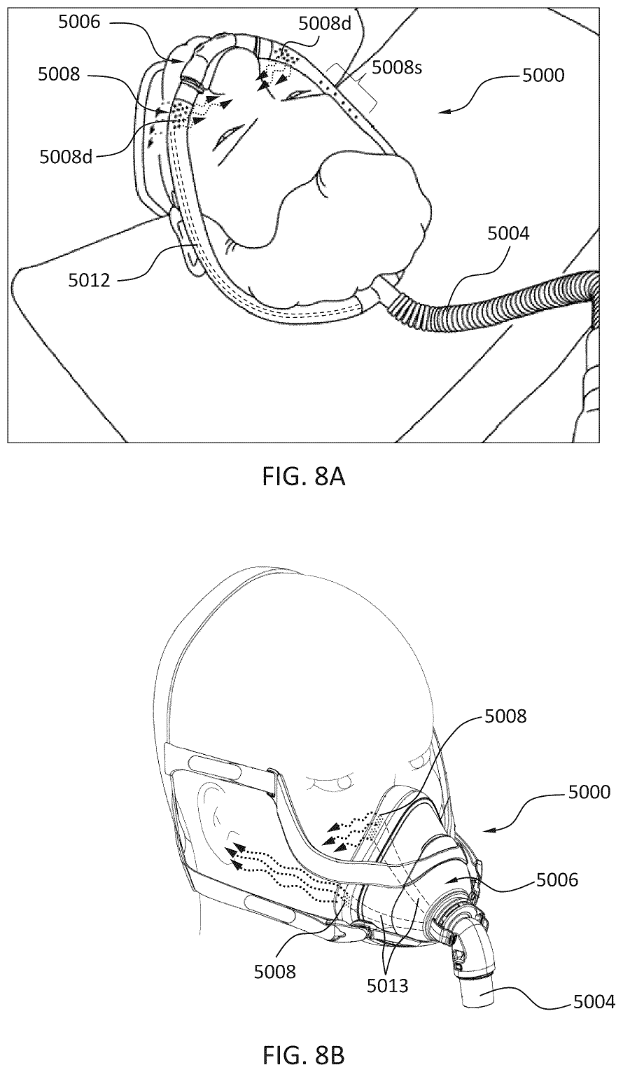

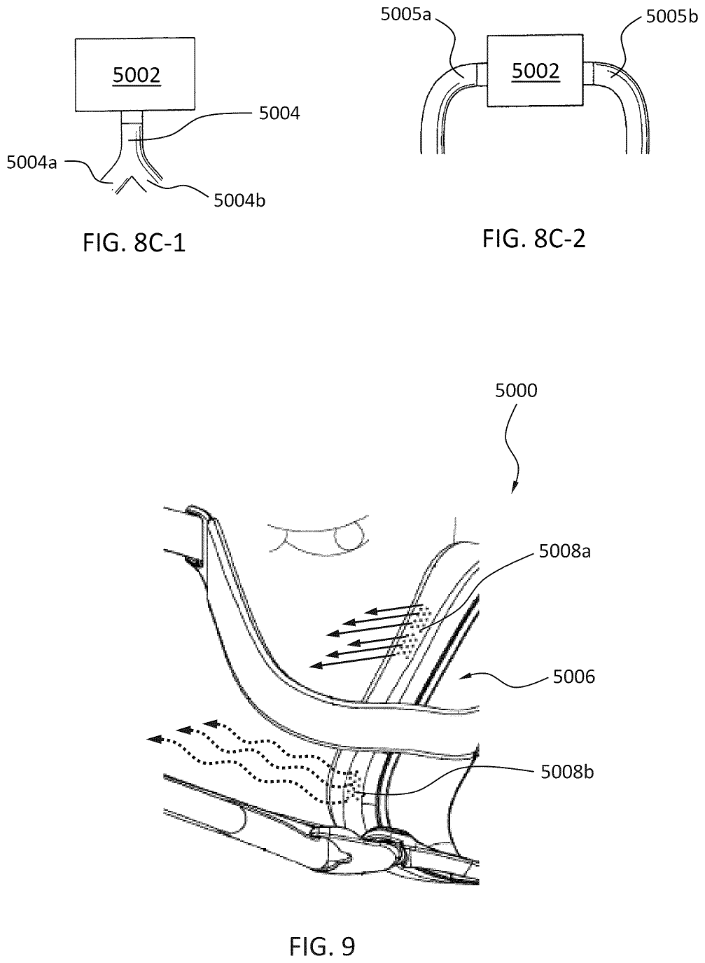

FIG. 8A shows an in-use apparatus for providing both respiratory pressure therapies and complementary therapies.

FIG. 8B shows an in-use apparatus for providing both respiratory pressure therapies and complementary therapies.

FIG. 8C-1 shows a schematic view of a dual-limbed tube for dividing airflow from a single tube connected to a flow generator; and FIG. 8C-2 shows a schematic view of a flow generator connected to two separate tubes for dividing airflow from a flow generator.

FIG. 9 shows an in-use apparatus for providing both respiratory pressure therapies and complementary therapies.

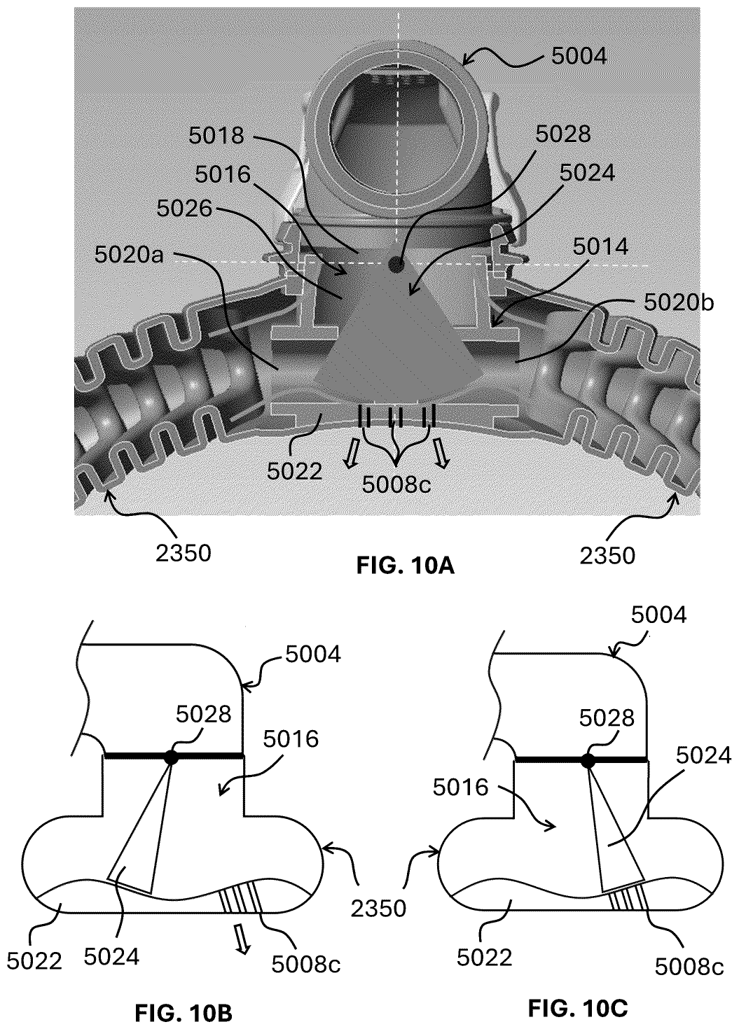

FIG. 10A-10C shows a first embodiment of a flow regulator for providing complementary therapy, where; FIG. 10A shows a sectional front view of the flow regulator; and FIGS. 10B and 10C show sectional side views of a schematic representation of the flow regulator.

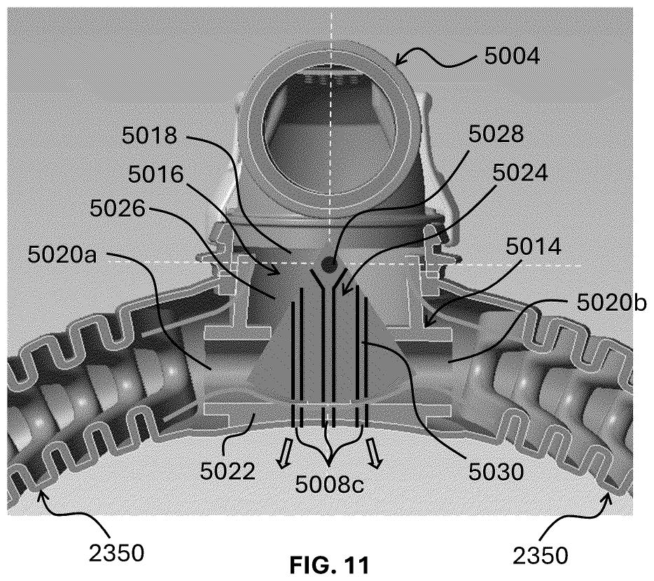

FIG. 11 shows a sectional front view of a second form of the first embodiment of a flow regulator.

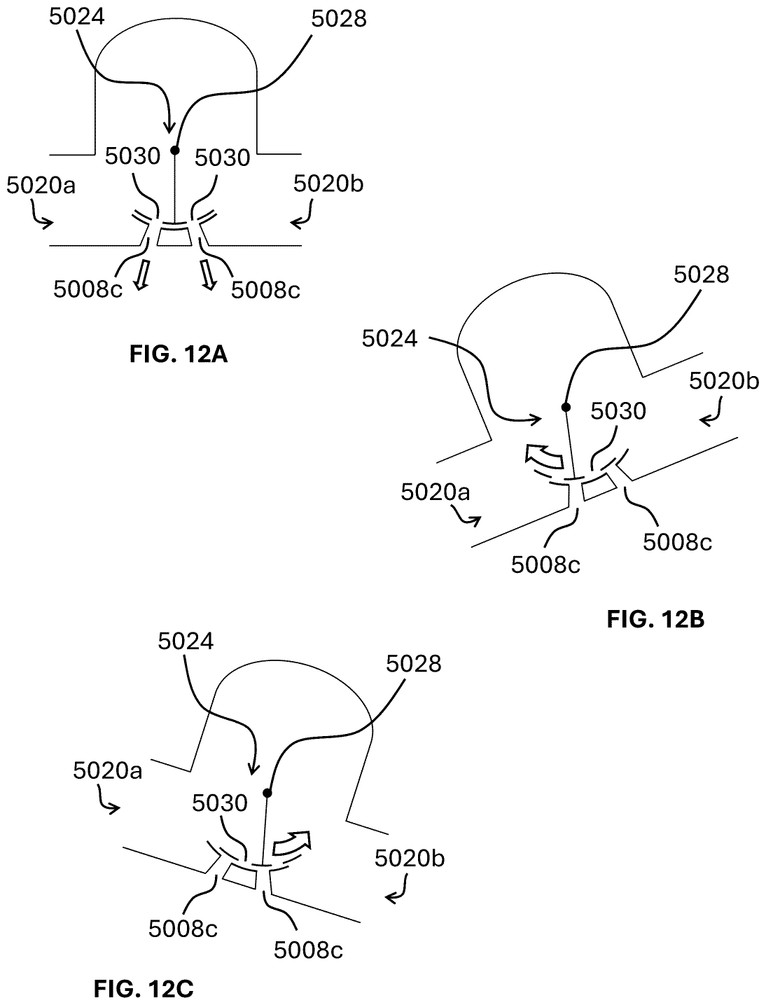

FIGS. 12A to 12C show front views of a schematic representation of the second form of the first embodiment of a flow regulator.

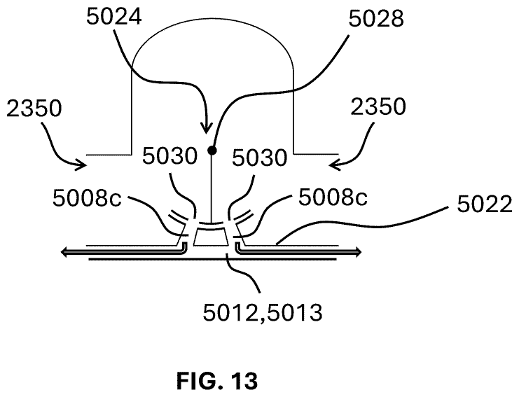

FIG. 13 shows a front view of a schematic representation of the second form of the first embodiment of a flow regulator.

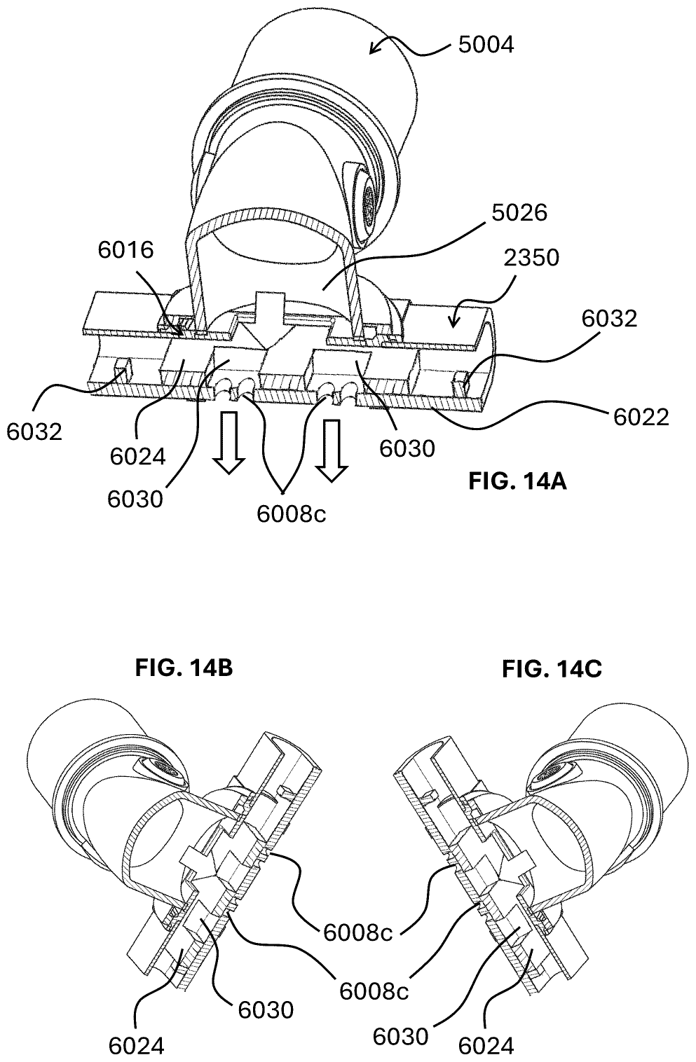

FIGS. 14A to 14C show front views of a second embodiment of a flow regulator for providing complementary therapy.

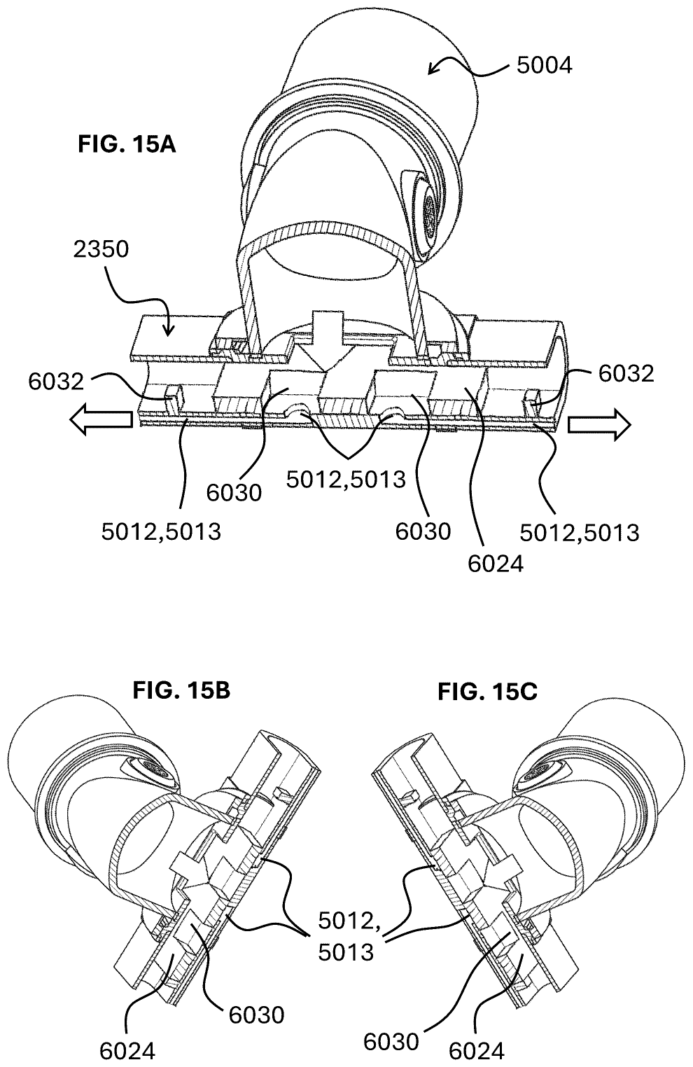

FIGS. 15A to 15C show front views of a second form of the second embodiment of a flow regulator for providing complementary therapy.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 3000 for supplying a flow of air to the patient 1000 via an air circuit 3170 and a patient interface 2000 or 2800.

### 4.3 Patient Interface

A non-invasive patient interface 2000, such as that shown in FIG. 3A, in accordance with one aspect of the present technology comprises the following functional aspects: a seal-forming structure 2100, a plenum chamber 2200, a positioning and stabilising structure 2300, a vent 2400, one form of connection port 2600 for connection to air circuit 3170, and a forehead support 2700. In some forms a functional aspect may be provided by one or more physical components. In some forms, one physical component may provide one or more functional aspects. In use the seal-forming structure 2100 is arranged to surround an entrance to the airways of the patient so as to maintain positive pressure at the entrance(s) to the airways of the patient 1000. The sealed patient interface 2000 is therefore suitable for delivery of positive pressure therapy.

The patient interface 2000 in accordance with one form of the present technology is constructed and arranged to be able to provide a supply of air at a positive pressure above the ambient, for example at least 2, 4, 6, 10, or 20 cmH2O with respect to ambient.

### 4.3.1 Seal-Forming Structure

In one form of the present technology, a seal-forming structure 2100 provides a target seal-forming region, and may additionally provide a cushioning function. The target seal-forming region is a region on the seal-forming structure 2100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 2100.

In certain forms of the present technology, the seal-forming structure 2100 is constructed from a biocompatible material, e.g. silicone rubber.

A seal-forming structure 2100 in accordance with the present technology may be constructed from a soft, flexible, resilient material such as silicone.

In certain forms of the present technology, a system is provided comprising more than one a seal-forming structure 2100, each being configured to correspond to a different size and/or shape range. For example the system may comprise one form of a seal-forming structure 2100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 2200 acting on its underside to urge it into tight sealing engagement with the face. The pressure assisted mechanism may act in conjunction with elastic tension in the positioning and stabilising structure.

In one form, the seal-forming structure 2100 comprises a sealing flange and a support flange. The sealing flange comprises a relatively thin member with a thickness of less than about 1 mm, for example about 0.25 mm to about 0.45 mm, which extends around the perimeter of the plenum chamber 2200. Support flange may be relatively thicker than the sealing flange. The support flange is disposed between the sealing flange and the marginal edge of the plenum chamber 2200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

4.3.1.2 Nose bridge or nose ridge region

In one form, the non-invasive patient interface 2000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 2000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 2000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 2000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nose-Only Masks

In one form, the patient interface 2000 comprises a seal-forming structure 2100 configured to seal around an entrance to the patient's nasal airways but not around the patient's mouth. The seal-forming structure 2100 may be configured to seal to the patient's lip superior. The patient interface 2000 may leave the patient's mouth uncovered. This patient interface 2000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 2100 configured to seal on the patient's face around the nose and over the bridge of the nose. A nasal mask may be generally triangular in shape. In one form, the non-invasive patient interface 2000 comprises a seal-forming structure 2100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 2000 shown in FIG. 1B has this type of seal-forming structure 2100. This patient interface 2000 may deliver a supply of air or breathable gas to both nares of patient 1000 through a single orifice.

Another form of nose-only mask may seal around an inferior periphery of the patient's nose without engaging the user's nasal ridge. This type of patient interface 2000 may be identified as a “nasal cradle” mask and the seal-forming structure 2100 may be identified as a “nasal cradle cushion”, for example. In one form, for example as shown in FIG. 3F, the seal-forming structure 2100 is configured to form a seal in use with inferior surfaces of the nose around the nares. The seal-forming structure 2100 may be configured to seal around the patient's nares at an inferior periphery of the patient's nose including to an inferior and/or anterior surface of a pronasale region of the patient's nose and to the patient's nasal alae. The seal-forming structure 2100 may seal to the patient's lip superior. The shape of the seal-forming structure 2100 may be configured to match or closely follow the underside of the patient's nose and may not contact a nasal bridge region of the patient's nose or any portion of the patient's nose superior to the pronasale. In one form of nasal cradle cushion, the seal-forming structure 2100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 2100 may comprise a single opening to provide a flow or air or breathable gas to both of the patient's nares.

In some forms, a nose-only mask may comprise nasal pillows, described above.

### 4.3.1.8 Nose and Mouth Masks

In one form, the patient interface 2000 comprises a seal-forming structure 2100 configured to seal around an entrance to the patient's nasal airways and also around the patient's mouth. The seal-forming structure 2100 may be configured to seal to the patient's face proximate a chin region. This patient interface 2000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 2100 configured to seal on the patient's face around the nose, below the mouth and over the bridge of the nose. A nose-and-mouth mask may be generally triangular in shape. In one form the patient interface 2000 comprises a seal-forming structure 2100 that forms a seal in use to a patient's chin-region (which may include the patient's lip inferior and/or a region directly inferior to the lip inferior), to the patient's nose bridge or at least a portion of the nose ridge superior to the pronasale, and to check regions of the patient's face. The patient interface 2000 shown in FIG. 1C is of this type. This patient interface 2000 may deliver a supply of air or breathable gas to both nares and mouth of patient 1000 through a single orifice. This type of seal-forming structure 2100 may be referred to as a “nose-and-mouth cushion”.

In another form the patient interface 2000 comprises a seal-forming structure 2100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 2100 may also form a seal against a patient's lip superior. A patient interface 2000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 2000 may have a nasal portion and an oral portion, the nasal portion sealing to the patient's face at similar locations to a nasal cradle mask.

In a further form of nose and mouth mask, the patient interface 2000 may comprise a seal-forming structure 2100 having a nasal portion comprising nasal pillows and an oral portion configured to form a seal to the patient's face around the patient's mouth.

In some forms, the seal-forming structure 2100 may have a nasal portion that is separate and distinct from an oral portion. In other forms, a seal-forming structure 2100 may form a contiguous seal around the patient's nose and mouth.

It is to be understood that the above examples of different forms of patient interface 2000 do not constitute an exhaustive list of possible configurations. In some forms a patient interface 2000 may comprise a combination of different features of the above described examples of nose-only and nose and mouth masks.

### 4.3.2 Plenum Chamber

The plenum chamber 2200 has a perimeter that is shaped to be complementary to the surface contour of the face of an average person in the region where a seal will form in use. In use, a marginal edge of the plenum chamber 2200 is positioned in close proximity to an adjacent surface of the face. Actual contact with the face is provided by the seal-forming structure 2100. The seal-forming structure 2100 may extend in use about the entire perimeter of the plenum chamber 2200. In some forms, the plenum chamber 2200 and the seal-forming structure 2100 are formed from a single homogeneous piece of material.

In certain forms of the present technology, the plenum chamber 2200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 2200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 2200 is constructed from a translucent material. The use of a translucent material can reduce the obtrusiveness of the patient interface, and help improve compliance with therapy.

In some forms, the plenum chamber 2200 is constructed from a rigid material such as polycarbonate. The rigid material may provide support to the seal-forming structure.

In some forms, the plenum chamber 2200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 2200 may be made from a material having Young's modulus of 0.1 GPa or lower, for example rubber. In other forms of the technology the plenum chamber 2200 may be made from a material having a Young's modulus of 0.7 MPa or less, for example between 0.7 MPa and 0.3 MPa. An example of such a material is silicone.

### 4.3.3 Positioning and Stabilising Structure

The seal-forming structure 2100 of the patient interface 2000 of the present technology may be held in sealing position in use by the positioning and stabilising structure 2300. The positioning and stabilising structure 2300 may comprise and function as “headgear” since it engages the patient's head in order to hold the patient interface 2000 in a sealing position. Examples of a positioning and stabilising structure may be shown in FIG. 3A.

In one form the positioning and stabilising structure 2300 provides a retention force at least sufficient to overcome the effect of the positive pressure in the plenum chamber 2200 to lift off the face (i.e., Fₚₗₑₙᵤₘ).

In one form the positioning and stabilising structure 2300 provides a retention force to overcome the effect of the gravitational force on the patient interface 2000.

In one form of the present technology, a positioning and stabilising structure 2300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 2300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 2300 comprises a strap that is bendable and e.g. non-rigid. An advantage of this aspect is that the strap is more comfortable for a patient to lie upon while the patient is sleeping.

In certain forms of the present technology, a positioning and stabilising structure 2300 comprises a strap constructed to be breathable to allow moisture vapour to be transmitted through the strap,

In certain forms of the present technology, a system is provided comprising more than one positioning and stabilising structure 2300, each being configured to provide a retaining force to correspond to a different size and/or shape range. For example the system may comprise one form of positioning and stabilising structure 2300 suitable for a large sized head, but not a small sized head, and another. suitable for a small sized head, but not a large sized head.

### 4.3.3.1 Conduit Headgear

### 4.3.3.1.1 Conduit Headgear Tubes

In some forms of the present technology, the positioning and stabilising structure 2300 comprises one or more headgear tubes 2350 that deliver pressurised air received from a conduit forming part of the air circuit 3170 from the RPT device to the patient's airways, for example through the plenum chamber 2200 and seal-forming structure 2100. In the form of the present technology illustrated in FIG. 3F, the positioning and stabilising structure 2300 comprises two tubes 2350 that deliver air to the plenum chamber 2200 from the air circuit 3170. The tubes 2350 are configured to position and stabilise the seal-forming structure 2100 of the patient interface 2000 at the appropriate part of the patient's face (for example, the nose and/or mouth) in use. This allows the conduit of air circuit 3170 providing the flow of pressurised air to connect to a connection port 2600 of the patient interface in a position other than in front of the patient's face, for example on top of the patient's head.

In the form of the present technology illustrated in FIG. 3F, the positioning and stabilising structure 2300 comprises two tubes 2350, each tube 2350 being positioned in use on a different side of the patient's head and extending across the respective cheek region, above the respective ear (superior to the otobasion superior on the patient's head) to the elbow 2610 on top of the head of the patient 1000. This form of technology may be advantageous because, if a patient sleeps with their head on its side and one of the tubes 2350 is compressed to block or partially block the flow of gas along the tube 2350, the other tube 2350 remains open to supply pressurised gas to the patient. In other examples of the technology, the patient interface 2000 may comprise a different number of tubes, for example one tube, or two or more tubes.

In one example in which the patient interface has one tube 2350, the single tube 2350 is positioned on one side of the patient's head in use (e.g. across one cheek region) and a strap forms part of the positioning and stabilising structure 2300 and is positioned on the other side of the patient's head in use (e.g. across the other region) to assist in securing the patient interface 2000 on the patient's head. For example, the tube 2350 and the strap may each be under tension in use in order to assist in maintaining the seal-forming structure 2100 in a sealing position.

In one form, the tube 2350 may be at least partially extensible so that the tube 2350 and the strap may adjust substantially equal lengths when worn by a patient. This may allow for substantially symmetrical adjustments between the tube 2350 and the strap so that the seal-forming structure remains substantially in the middle.

In the form of the technology shown in FIG. 3F, the two tubes 2350 are fluidly connected at superior ends to each other and to the connection port 2600. In some examples, the two tubes 2350 are integrally formed while in other examples the tubes 2350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 2350. Additionally, the T-shaped connector may have a third arm or opening providing the connection port 2600 for fluid connection to the air circuit 3170 in use. The opening may be an inlet 2332 (see e.g., 7C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 2300.

In some forms, at least one of the arms of the T-shaped connector (or Y-shaped connector) may be at least partially rigidised. This may assist in maintaining the shape of the connector so that bending of the connector does not close the airflow path.

The tubes 2350 may be formed from a flexible material, such as an elastomer, e.g. silicone or TPE, and/or from one or more textile and/or foam materials. The tubes 2350 may have a preformed shape and may be able to be bent or moved into another shape upon application of a force but may return to the original preformed shape in the absence of said force. The tubes 2350 may be generally arcuate or curved in a shape approximating the contours of a patient's head between the top of the head and the nasal or oral region.

In some examples, the one or more tubes 2350 are crush resistant to resist being blocked if crushed during use, for example if squashed between a patient's head and pillow, especially if there is only one tube 2350. The tubes 2350 may be formed with a sufficient structural stiffness to resist crushing or may be as described in U.S. Pat. No. 6,044,844, the contents of which are incorporated herein by reference.

Each tube 2350 may be configured to receive a flow of air from the connection port 2600 on top of the patient's head and to deliver the flow of air to the seal-forming structure 2100 at the entrance of the patient's airways. In the example shown in FIG. 3F, each tube 2350 lies in use on a path extending from the plenum chamber 2200 across the patient's cheek region and superior to the patient's ear to the elbow 2610. For example, a portion of each tube 2350 proximate the plenum chamber 2200 may overlie a maxilla region of the patient's head in use. Another portion of each tube 2350 may overlie a region of the patient's head superior to an otobasion superior of the patient's head. Each of the tubes 2350 may also lie over the patient's sphenoid bone and/or temporal bone and either or both of the patient's frontal bone and parietal bone. The elbow 2610 may be located in use over the patient's parietal bone, over the frontal bone and/or over the junction therebetween (e.g. the coronal suture).

In certain forms of the present technology the patient interface 2000 is configured such that the connection port 2600 can be positioned in a range of positions across the top of the patient's head so that the patient interface 2000 can be positioned as appropriate for the comfort or fit of an individual patient. In some examples, the headgear tubes 2350 are configured to allow movement of an upper portion of the patient interface 2000 (e.g. a connection port 2600) with respect to a lower portion of the patient interface 2000 (e.g. a plenum chamber 2200). That is, the connection port 2600 may be at least partially decoupled from the plenum chamber 2200. In this way, the seal-forming structure 2100 may form an effective seal with the patient's face irrespective of the position of the connection port 2600 (at least within a predetermined range of positions) on the patient's head.

As described above, in some examples of the present technology the patient interface 2000 comprises a seal-forming structure 2100 in the form of a cradle cushion which lies generally under the nose and seals to an inferior periphery of the nose (e.g. an under-the-nose cushion). The positioning and stabilising structure 2300, including the tubes 2350 may be structured and arranged to pull the seal-forming structure 2100 into the patient's face under the nose with a sealing force in a posterior and superior direction (e.g. a posterosuperior direction). A sealing force with a posterosuperior direction may cause the seal-forming structure 2100 to form a good seal to both the inferior periphery of the patient's nose and anterior-facing surfaces of the patient's face, for example on either side of the patient's nose and the patient's lip superior.

### 4.3.3.1.2 Conduit Headgear Connection Port

In certain forms of the present technology, the patient interface 2000 may comprise a connection port 2600 located proximal to a superior, lateral or posterior portion of a patient's head. For example, in the form of the present technology illustrated in FIG. 3F, the connection port 2600 is located on top of the patient's head (e.g. at a superior location with respect to the patient's head). In this example the patient interface 2000 comprises an elbow 2610 forming the connection port 2600. The elbow 2610 may be configured to fluidly connect with a conduit of an air circuit 3170. The elbow 2610 may be configured to swivel with respect to the positioning and stabilising structure 2300 to at least partially decouple the conduit from the positioning and stabilising structure 2300. In some examples the elbow 2610 may be configured to swivel by rotation about a substantially vertical axis and, in some particular examples, by rotation about two or more axes. In some examples the elbow may comprise or be connected to the tubes 2350 by a ball-and-socket joint. The connection portion 2600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 4.3.3.1.3 Headgear Tube Fluid Connections

The two tubes 2350 are fluidly connected at their inferior ends to the plenum chamber 2200. In certain forms of the technology, the connection between the tubes 2350 and the plenum chamber 2200 is achieved by connection of two rigid connectors. The tubes 2350 and plenum chamber 2200 may be configured to enable the patient to easily connect the two components together in a reliable manner. The tubes 2350 and plenum chamber 2200 may be configured to provide tactile and/or audible feedback in the form of a ‘re-assuring click’ or a similar sound, so that the patient may easily know that each tube 2350 has been correctly connected to the plenum chamber 2200. In one form, the tubes 2350 are formed from a silicone or textile material and the inferior end of each of the silicone tubes 2350 is overmolded to a rigid connector made, for example, from polypropylene, polycarbonate, nylon or the like. The rigid connector on each tube 2350 may comprise a female mating feature configured to connect with a male mating feature on the plenum chamber 2200. Alternatively, the rigid connector on each tube 2350 may comprise a male mating feature configured to connect to a female mating feature on the plenum chamber 2200. In other examples the tubes 2350 may each comprise a male or female connector formed from a flexible material, such as silicone or TPE, for example the same material from which the tubes 2350 are formed.

In other examples a compression seal is used to connect each tube 2350 to the plenum chamber 2200. For example, a resiliently flexible (e.g. silicone) tube 2350 without a rigid connector may be configured to be squeezed to reduce its diameter so that it can be compressed into a port in the plenum chamber 2200 and the inherent resilience of the silicone pushes the tube 2350 outwards to seal the tube 2350 in the port in an air-tight manner. Alternatively, in a hard-to-hard type engagement between the tube 2350 and the plenum chamber 2200, each tube 2350 and/or plenum chamber 2200 may comprise a pressure activated seal, for example a peripheral sealing flange. When pressurised gas is supplied through the tubes 2350 the sealing flange may be urged against the join between the tubes and a circumferential surface around a port or connector of the plenum chamber 2200 to form or enhance a seal between the tube 2350 and plenum chamber 2200.

### 4.3.3.2 Headgear Straps

In some forms, the positioning and stabilising structure 2300 may include headgear 2302 with at least one strap which may be worn by the patient in order to assist in properly orienting the seal-forming structure 2100 against the patient's face (e.g., in order to limit or prevent leaks).

As described above, some forms of the headgear 2302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 2302.

In certain forms, the headgear 2302 may be at least partially extensible. For example, the headgear 2302 may include elastic, or a similar extensible material. For example, the entire headgear 2302 may be extensible or selected portions may be extensible (or more extensible than surrounding portions). This may allow the headgear 2302 to stretch while under tension, which may assist in providing a sealing force for the seal-forming structure 2100.

Two forms of the headgear, four-point headgear 2302-1 (see FIG. 3A) and two-point headgear 2302-2 (see FIG. 3F).

### 4.3.4 Vent

In one form, the patient interface 2000 includes a vent 2400 constructed and arranged to allow for the washout of exhaled gases, e.g. carbon dioxide.

In certain forms the vent 2400 is configured to allow a continuous vent flow from an interior of the plenum chamber 2200 to ambient whilst the pressure within the plenum chamber is positive with respect to ambient. The vent 2400 is configured such that the vent flow rate has a magnitude sufficient to reduce rebreathing of exhaled CO2 by the patient while maintaining the therapeutic pressure in the plenum chamber in use.

One form of vent 2400 in accordance with the present technology comprises a plurality of holes, for example, about 20 to about 80 holes, or about 40 to about 60 holes, or about 45 to about 55 holes.

The vent 2400 may be located in the plenum chamber 2200. Alternatively, the vent 2400 is located in a decoupling structure, e.g., a swivel.

### 4.3.5 Decoupling Structure(s)

In one form the patient interface 2000 includes at least one decoupling structure, for example, a swivel or a ball and socket.

### 4.3.6 Connection Port

Connection port 2600 allows for connection to the air circuit 3170.

### 4.3.7 Forehead Support

In one form, the patient interface 2000 includes a forehead support 2700.

### 4.3.8 Anti-Asphyxia Valve

In one form, the patient interface 2000 includes an anti-asphyxia valve.

### 4.3.9 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 2350 (see e.g., FIG. 3F). This may be referred to as a “tube up” configuration and may position a connection port at the top of the patient's head. In other example, the patient may receive the flow of pressurized air through a conduit connected to the plenum chamber 2200, for example through the connection port 2600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 4.4 RPT Device

An RPT device 3000 in accordance with one aspect of the present technology comprises mechanical, pneumatic, and/or electrical components and is configured to execute one or more algorithms 3300, such as any of the methods, in whole or in part, described herein. The RPT device 3000 may be configured to generate a flow of air for delivery to a patient's airways, such as to treat one or more of the respiratory conditions described elsewhere in the present document.

The RPT device may have an external housing 3010, formed in two parts, an upper portion 3012 and a lower portion 3014. Furthermore, the external housing 3010 may include one or more panel(s) 3015. The RPT device 3000 comprises a chassis 3016 that supports one or more internal components of the RPT device 3000. The RPT device 3000 may include a handle 3018.

The pneumatic path of the RPT device 3000 may comprise one or more air path items, e.g., an inlet air filter 3112, an inlet muffler 3122, a pressure generator 3140 capable of supplying air at positive pressure (e.g., a blower 3142), an outlet muffler 3124 and one or more transducers 3270, such as pressure sensors 3272 and flow rate sensors 3274.

One or more of the air path items may be located within a removable unitary structure which will be referred to as a pneumatic block 3020. The pneumatic block 3020 may be located within the external housing 3010. In one form a pneumatic block 3020 is supported by, or formed as part of the chassis 3016.

As shown in FIG. 4C, the RPT device 3000 may have an electrical power supply 3210, one or more input devices 3220, a central controller 3230, a therapy device controller 3240, a pressure generator 3140, one or more protection circuits 3250, memory 3260, transducers 3270, data communication interface 3280 and one or more output devices 3290. Electrical components 3200 may be mounted on a single Printed Circuit Board Assembly (PCBA) 3202. In an alternative form, the RPT device 3000 may include more than one PCBA 3202.

### 4.4.1 RPT Device Mechanical & Pneumatic Components

An RPT device may comprise one or more of the following components in an integral unit. In an alternative form, one or more of the following components may be located as respective separate units.

### 4.4.1.1 Air Filter(s)

An RPT device in accordance with one form of the present technology may include an air filter 3110, or a plurality of air filters 3110.

In one form illustrated in FIG. 4B, an inlet air filter 3112 is located at the beginning of the pneumatic path upstream of a pressure generator 3140.

In one form illustrated in FIG. 4B, an outlet air filter 3114, for example an antibacterial filter, is located between an outlet of the pneumatic block 3020 and a patient interface 2000 or 2800.

### 4.4.1.2 Muffler(s)

An RPT device in accordance with one form of the present technology may include a muffler 3120, or a plurality of mufflers 3120.

In one form of the present technology (see e.g., FIG. 4B), an inlet muffler 3122 is located in the pneumatic path upstream of a pressure generator 3140.

In one form of the present technology, an outlet muffler 3124 is located in the pneumatic path between the pressure generator 3140 and a patient interface 2000 or 2800.

### 4.4.1.3 Pressure Generator

In one form of the present technology, a pressure generator 3140 for producing a flow, or a supply, of air at positive pressure is a controllable blower 3142. For example, the blower 3142 may include a brushless DC motor 3144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 3140 may be under the control of the therapy device controller 3240.

In other forms, a pressure generator 3140 may be a piston-driven pump, a pressure regulator connected to a high pressure source (e.g. compressed air reservoir), or a bellows.

### 4.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 4B), one or more transducers 3270 are located upstream and/or downstream of the pressure generator 3140. The one or more transducers 3270 may be constructed and arranged to generate signals representing properties of the flow of air such as a flow rate, a pressure or a temperature at that point in the pneumatic path.

In one form of the present technology, one or more transducers 3270 may be located proximate to the patient interface 2000 or 2800.

In one form, a signal from a transducer 3270 may be filtered, such as by low-pass, high-pass or band-pass filtering.

### 4.4.1.4.1 Flow Rate Sensor

A flow rate sensor 3274 in accordance with the present technology may be based on a differential pressure transducer, for example, an SDP600 Series differential pressure transducer from SENSIRION.

In one form, a signal generated by the flow rate sensor 3274 and representing a flow rate is received by the central controller 3230.

### 4.4.1.4.2 Pressure Sensor

A pressure sensor 3272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 3272 and representing a pressure is received by the central controller 3230.

### 4.4.1.4.3 Motor Speed Transducer

In one form of the present technology a motor speed transducer 3276 is used to determine a rotational velocity of the motor 3144 and/or the blower 3142. A motor speed signal from the motor speed transducer 3276 may be provided to the therapy device controller 3240. The motor speed transducer 3276 may, for example, be a speed sensor, such as a Hall effect sensor.

### 4.4.1.5 Anti-Spill Back Valve

As shown in FIG. 4B, one form of the present technology, an anti-spill back valve 3160 is located between the humidifier 4000 and the pneumatic block 3020. The anti-spill back valve is constructed and arranged to reduce the risk that water will flow upstream from the humidifier 4000, for example to the motor 3144.

### 4.4.2 RPT Device Electrical Components

### 4.4.2.1 Power Supply

A power supply 3210 may be located internal or external of the external housing 3010 of the RPT device 3000.

In one form of the present technology, power supply 3210 provides electrical power to the RPT device 3000 only. In another form of the present technology, power supply 3210 provides electrical power to both RPT device 3000 and humidifier 4000.

As illustrated in FIG. 4C-1, the power supply 3210 may provide electrical power to the input device 3220, the central controller 3230, the output device 3290, and the pressure generator 3140. The power supply 3210 may also provide electric energy to other components of the RPT device 3000 (or the humidifier 4000, as described above).

### 4.4.2.2 Input Devices

In one form of the present technology, an RPT device 3000 includes one or more input devices 3220 in the form of buttons, switches or dials to allow a person to interact with the device. The buttons, switches or dials may be physical devices, or software devices accessible via a touch screen. The buttons, switches or dials may, in one form, be physically connected to the external housing 3010, or may, in another form, be in wireless communication with a receiver that is in electrical connection to the central controller 3230.

In one form, the input device 3220 may be constructed and arranged to allow a person to select a value and/or a menu option.

### 4.4.2.3 Central Controller

In one form of the present technology, the central controller 3230 is one or a plurality of processors suitable to control an RPT device 3000. The central controller 3230 is show in FIGS. 4C and 4C-1.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 3230 is a dedicated electronic circuit.

In one form, the central controller 3230 is an application-specific integrated circuit. In another form, the central controller 3230 comprises discrete electronic components.

The central controller 3230 may be configured to receive input signal(s) from one or more transducers 3270, one or more input devices 3220, and/or the humidifier 4000.

The central controller 3230 may be configured to provide output signal(s) to one or more of an output device 3290, a pressure generator 3140, a therapy device controller 3240, a data communication interface 3280, and/or the humidifier 4000.

In some forms of the present technology, the central controller 3230 is configured to implement the one or more methodologies described herein, such as the one or more algorithms 3300 which may be implemented with processor-control instructions, expressed as computer programs stored in a non-transitory computer readable storage medium, such as memory 3260. In some forms of the present technology, the central controller 3230 may be integrated with an RPT device 3000. However, in some forms of the present technology, some methodologies may be performed by a remotely located device. For example, the remotely located device may determine control settings for a ventilator or detect respiratory related events by analysis of stored data such as from any of the sensors described herein.

### 4.4.2.4 Clock

The RPT device 3000 may include a clock 3232 that is connected to the central controller 3230.

### 4.4.2.5 Therapy Device Controller

In one form of the present technology, therapy device controller 3240 is a therapy control module 3330 that forms part of the algorithms 3300 executed by the central controller 3230.

In one form of the present technology, therapy device controller 3240 is a dedicated motor control integrated circuit. For example, in one form a MC33035 brushless DC motor controller, manufactured by ONSEMI is used.

### 4.4.2.6 Protection Circuits

The one or more protection circuits 3250 in accordance with the present technology may comprise an electrical protection circuit, a temperature and/or pressure safety circuit.

### 4.4.2.7 Memory

In accordance with one form of the present technology the RPT device 3000 includes memory 3260, e.g., non-volatile memory. In some forms, memory 3260 may include battery powered static RAM. In some forms, memory 3260 may include volatile RAM.

Memory 3260 may be located on the PCBA 3202. Memory 3260 may be in the form of EEPROM, or NAND flash.

Additionally, or alternatively, RPT device 3000 includes a removable form of memory 3260, for example a memory card made in accordance with the Secure Digital (SD) standard.

In one form of the present technology, the memory 3260 acts as a non-transitory computer readable storage medium on which is stored computer program instructions expressing the one or more methodologies described herein, such as the one or more algorithms 3300.

### 4.4.2.8 Data Communication Systems

In one form of the present technology, a data communication interface 3280 is provided, and is connected to the central controller 3230 (see e.g., FIG. 4C). Data communication interface 3280 may be connectable to a remote external communication network 3282 and/or a local external communication network 3284. The remote external communication network 3282 may be connectable to a remote external device 3286. The local external communication network 3284 may be connectable to a local external device 3288.

In one form, data communication interface 3280 is part of the central controller 3230. In another form, data communication interface 3280 is separate from the central controller 3230, and may comprise an integrated circuit or a processor.

In one form, remote external communication network 3282 is the Internet. The data communication interface 3280 may use wired communication (e.g. via Ethernet, or optical fibre) or a wireless protocol (e.g. CDMA, GSM, LTE) to connect to the Internet.

In one form, local external communication network 3284 utilises one or more communication standards, such as Bluetooth, or a consumer infrared protocol.

In one form, remote external device 3286 is one or more computers, for example a cluster of networked computers. In one form, remote external device 3286 may be virtual computers, rather than physical computers. In either case, such a remote external device 3286 may be accessible to an appropriately authorised person such as a clinician.

The local external device 3288 may be a personal computer, mobile phone, tablet or remote control.

### 4.4.2.9 Output Devices Including Optional Display, Alarms

An output device 3290 in accordance with the present technology may take the form of one or more of a visual, audio and haptic unit. A visual display may be a Liquid Crystal Display (LCD) or Light Emitting Diode (LED) display.

### 4.4.2.9.1 Display Driver

A display driver 3292 receives as an input the characters, symbols, or images intended for display on the display 3294, and converts them to commands that cause the display 3294 to display those characters, symbols, or images.

### 4.4.2.9.2 Display

A display 3294 is configured to visually display characters, symbols, or images in response to commands received from the display driver 3292. For example, the display 3294 may be an eight-segment display, in which case the display driver 3292 converts each character or symbol, such as the figure “0”, to eight logical signals indicating whether the eight respective segments are to be activated to display a particular character or symbol.

### 4.5 Air Circuit

An air circuit 3170 in accordance with an aspect of the present technology is a conduit or a tube constructed and arranged to allow, in use, a flow of air to travel between two components such as RPT device 3000 and the patient interface 2000 or 2800.

In particular, the air circuit 3170 may be in fluid connection with the outlet of the pneumatic block 3020 and the patient interface. The air circuit may be referred to as an air delivery tube. In some cases there may be separate limbs of the circuit for inhalation and exhalation. In other cases a single limb is used.

In some forms, the air circuit 3170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 3170. The heating element may be in communication with a controller such as a central controller 3230. One example of an air circuit 3170 comprising a heated wire circuit is described in U.S. Pat. No. 8,733,349, which is incorporated herewithin in its entirety by reference.

### 4.6 Humidifier

### 4.6.1 Humidifier Overview

In one form of the present technology there is provided a humidifier 4000 (e.g. as shown in FIG. 5A) to change the absolute humidity of air or gas for delivery to a patient relative to ambient air. Typically, the humidifier 4000 is used to increase the absolute humidity and increase the temperature of the flow of air (relative to ambient air) before delivery to the patient's airways.

The humidifier 4000 may comprise a humidifier reservoir 4110, a humidifier inlet 4002 to receive a flow of air, and a humidifier outlet 4004 to deliver a humidified flow of air. In some forms, as shown in FIG. 5A and FIG. 5B, an inlet and an outlet of the humidifier reservoir 4110 may be the humidifier inlet 4002 and the humidifier outlet 4004 respectively. The humidifier 4000 may further comprise a humidifier base 4006, which may be adapted to receive the humidifier reservoir 4110 and comprise a heating element 4240.

### 4.6.2 Humidifier Components

### 4.6.2.1 Water Reservoir

According to one arrangement, the humidifier 4000 may comprise a water reservoir 4110 configured to hold, or retain, a volume of liquid (e.g. water) to be evaporated for humidification of the flow of air. The water reservoir 4110 may be configured to hold a predetermined maximum volume of water in order to provide adequate humidification for at least the duration of a respiratory therapy session, such as one evening of sleep. Typically, the reservoir 4110 is configured to hold several hundred millilitres of water, e.g. 300 millilitres (ml), 325 ml, 350 ml or 400 ml. In other forms, the humidifier 4000 may be configured to receive a supply of water from an external water source such as a building's water supply system.

According to one aspect, the water reservoir 4110 is configured to add humidity to a flow of air from the RPT device 3000 as the flow of air travels therethrough. In one form, the water reservoir 4110 may be configured to encourage the flow of air to travel in a tortuous path through the reservoir 4110 while in contact with the volume of water therein.

According to one form, the reservoir 4110 may be removable from the humidifier 4000, for example in a lateral direction as shown in FIG. 5A and FIG. 5B.

The reservoir 4110 may also be configured to discourage egress of liquid therefrom, such as when the reservoir 4110 is displaced and/or rotated from its normal, working orientation, such as through any apertures and/or in between its sub-components. As the flow of air to be humidified by the humidifier 4000 is typically pressurised, the reservoir 4110 may also be configured to prevent losses in pneumatic pressure through leak and/or flow impedance.

### 4.6.2.2 Conductive Portion

According to one arrangement, the reservoir 4110 comprises a conductive portion 4120 configured to allow efficient transfer of heat from the heating element 4240 to the volume of liquid in the reservoir 4110. In one form, the conductive portion 4120 may be arranged as a plate, although other shapes may also be suitable. All or a part of the conductive portion 4120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 4.6.2.3 Humidifier Reservoir Dock

In one form, the humidifier 4000 may comprise a humidifier reservoir dock 4130 (as shown in FIG. 5B) configured to receive the humidifier reservoir 4110. In some arrangements, the humidifier reservoir dock 4130 may comprise a locking feature such as a locking lever 4135 configured to retain the reservoir 4110 in the humidifier reservoir dock 4130.

### 4.6.2.4 Water Level Indicator

The humidifier reservoir 4110 may comprise a water level indicator 4150 as shown in FIG. 5A-5B. In some forms, the water level indicator 4150 may provide one or more indications to a user such as the patient 1000 or a care giver regarding a quantity of the volume of water in the humidifier reservoir 4110. The one or more indications provided by the water level indicator 4150 may include an indication of a maximum, predetermined volume of water, any portions thereof, such as 25%, 50% or 75% or volumes such as 200 ml, 300 ml or 400 ml.

### 4.7 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Tᵢ 1.6 s, peak inspiratory flow rate Qₚₑₐₖ 0.4 L/s, exhalation time Tₑ 2.4 s, peak expiratory flow rate Qₚₑₐₖ−0.5 L/s. The total duration of the breath, Tₜₒₜ, is about 4 s. The person typically breathes at a rate of about 15 breaths per minute (BPM), with Ventilation Vent about 7.5 L/min. A typical duty cycle, the ratio of Tᵢ to Tₜₒₜ, is about 40%.

### 4.8 Complementary Therapy

In an embodiment of the present technology, airflow generated for respiratory pressure therapies, e.g., CPAP, may also be utilised to provide a complementary therapy. As set forth in more detail below, at least a portion of the airflow generated for respiratory therapy may also be used for the complementary therapy. In this regard, the complementary therapy may be an airflow derived, e.g., diverted, from an air pressure generator and configured to provide an alternative type of therapy, i.e., to be additional to and different from the existing therapy (i.e., respiratory therapy) being provided.

Referring to FIG. 7, an apparatus 5000 is shown for utilising the airflow generated by the air pressure generator, e.g., a flow generator 5002, supplied to the patient via a pressurised air conduit, e.g., tube 5004, leading to a patient interface, e.g., sealing structure (such as full-faced mask 5006 as shown in FIG. 8B), or sealing structure (such as nasal mask 5006 as shown in FIG. 8A and headgear tubing 5012). The mask 5006 (in any form) can be a seal-forming structure as set forth previously, being configured to provide a seal around the patient's airway.

In one form of the technology, a complementary flow device 5008 is provided, at least in part, on the patient interface for adapting a portion of the airflow from the flow generator 5002 for use as either an active complementary therapy or a passive complementary therapy (as set forth in more detail below).

The patient interface, complementary flow device and/or the flow generator 5002 may also be connected to a sensory monitoring and stimulation unit 5010. As set forth in more detail later, the sensory monitoring and stimulation unit 5010 may be implemented together with a controller (not shown) to control how the active and passive types of complementary therapy are delivered to the patient.

As set forth above, in some forms the complementary therapy may be active in nature, whereby the portion of the air flow from the complementary flow device, is configured for treating respiratory disorders, e.g., OSA. An example of the active complementary airflow can be positional therapy, wherein the airflow is diverted to stimulate a patient to change their sleeping position.

In other forms, the complementary therapy may be passive in nature, whereby the complementary airflow is configured to improve the sleeping environment of the patient. The sleeping environment may include the air temperature of a location, e.g., a room in which the patient is sleeping, ambient light, noise, air humidity, air pollutants (i.e., pollen, allergens etc.), etc.

Thus, the passive complementary therapy may involve the use of diverted airflow to move air around the patient (by e.g., blowing air across the patient's skin) so as to cool the patient during sleep.

Referring to FIGS. 8A and 8B, an in-use apparatus 5000 is shown comprising the patient interface for delivery of a supply of breathable gas to an entrance of the patient's airways via the tube 5004. In FIG. 8A, the patient interface includes nasal pillows mask 5006 and hear gear tubing 5012. In FIG. 8B, the patient interface includes full-faced mask 5006. The patient interface (in either form) incorporates the complementary flow device 5008 for delivery of the complementary airflow. In either case, the patient interface may be configured to deliver the complementary therapy as active or passive therapy.

### 4.8.1 Tubing

The apparatus 5000 may include a single tube 5004 extending between the flow generator 5002 and the mask 5006. As set forth previously, the tube 5004 may deliver pressurised airflow to the mask 5006 (as shown in FIG. 8B). This pressurised airflow can be considered a primary airflow or stream, i.e., having a first portion of the flow of air for providing the respiratory therapy and including a second portion that may be diverted from the primary airflow stream to be used in complementary therapy. That is, the complementary flow device may divert a secondary airflow, i.e., a second portion of the flow of air, from the primary airflow generated by the flow generator 5002. This secondary airflow is directed away from the patient's airways.

In some forms, the complementary flow device 5008 may be provided in an external wall of the mask, i.e., the plenum chamber, for directing the airflow received from the tubing onto the patient. In this form, the complementary flow device 5008 may divert the airflow prior to being delivered within the plenum chamber into a source of airflow for: i) the existing respiratory therapy; and ii) the complementary therapy.

As set forth in more detail later, the airflow may be diverted from the single tube 5004 by airflow routing. Such an arrangement is shown in FIG. 8B. This routing 5013 may be configured in the patient interface to direct air from the tube 5004. In the form where a single tube is connected between the flow generator 5002 and the mask 5006, the airflow may be diverted to the routing (from the tube 5004) before the airflow enters the plenum chamber of the full-faced mask 5006. That is, airflow from the flow generator 5002 is diverted into the routing before the airflow can mix with e.g., exhaled air of the patient contained in the mask 5006.

Advantageously, diverting the airflow in this way, i.e., avoiding mixing with air in the mask, can assist in maintaining a lower air temperature and humidity in the air delivered for the complementary therapy. In other words, if the primary airflow (i.e., generated by the flow generator) enters the mask before it is diverted into the secondary airflow (i.e., diverted into the routing), the primary airflow will mix with the exhaled air of the patient within the mask (i.e., plenum chamber) to warm and humidify the air before it is diverted into the secondary airflow.

In some forms of the apparatus 5000, the secondary airflow directed to the patient may be preferably warmed and humidified. In other forms, the secondary airflow may be preferably cooled, or remain generally at an ambient room temperature prior to contacting the patient's skin. As set forth in more detail later, the temperature and humidity of the secondary airflow, i.e., the airflow contacting the patient's skin, may be chosen according to the type of complementary therapy administered. For example, if the patient's body temperature is determined to be too high, the secondary airflow may be preferably cooled. Alternatively, if the ambient air temperature (i.e., around the patient) is determined to be cold, the secondary airflow may be warmed for e.g., gently stimulating the patient to e.g. wake up at a predetermined time. In some forms, the secondary airflow is directed to, or around, the patient without any further conditioning.

In other forms, the apparatus 5000 may include separate airflows, e.g., two, three, etc., sources of airflow. For example, and as illustrated in FIGS. 8C-1 and 8C-2, two tubes 5004a, 5004b, 5005a, 5005b can be provided for delivering two separate airflows to the patient interface. In the form shown in FIG. 8C-1, a dual-limbed tube may be provided for dividing airflow from a single tube 5004 into two separate airflows to be delivered to the patient. In this form of dual-limbed tube, the single tube 5004 connected to the flow generator 5002 can be divided into branches 5004a, 5004 for directing the separate airflows to the patient.

In the form shown in FIG. 8C-2, two separate tubes 5005a, 5005b are configured to extend between the flow generator 5002 and patient interface, each having a separate airflow output generated by the flow generator 5002. In this form, each tube is separately connected to the flow generator, such that the flow generator can be configured to generate and direct airflow of e.g., different velocity, to the mask via the separate tubes.

In this form, one of the tubes provided may be configured to deliver the prescribed therapeutic pressure to the patient. The other of the tubes may form part of, or be adapted to connect with, the complementary flow device 5008, whereby the device 5008 is configured to deliver the complementary airflow to the patient.

Advantageously, the separate airflows through each tube may be conditioned differently. For example, the humidity, temperature, pressure, etc may be different between the two tubes, so as to provide different types of airflow to the patient. In the case of the dual-limbed tube of FIG. 8C-1, one of the tubes may be provided with a heating element for heating the airflow delivered to the mask 5006, while the other of the tubes may not be heated, so as to deliver a cooler (and drier) airflow for use with the complementary therapy.

In some forms of the of the separate tubes, i.e., FIG. 8C-2, each tube may be provided with a different source of airflow, whereby the flow generator is configured to provide the two tubes with two corresponding, independent sources of airflow. For example, each airflow (delivered through each tube) may be supplied by a different motor. In this case, each motor may independently control airflow through each tube. Advantageously, this can minimise the magnitude of electrical load placed on a single motor supplying both the existing respiratory therapy and the complementary therapy with a separate source of airflow.

In some forms the two tubes may be configured as a concentric tube having an inner and outer tube. In this form, the inner tube may be configured to deliver the primary airflow to the patient interface, i.e., for respiratory therapy, and the outer tube may be configured to deliver a separate, secondary airflow for the complementary therapy. In this regard, the secondary flow may be a coterminous flow (i.e., through the inner tube contained within the outer tube).

In either form, i.e., having the single tube 5004, two separate or dual-limbed tubes 5004a, 5004b, 5005a, 5005b, or the concentric tube, the patient interface may also be provided with the airflow routing (as previously mentioned) for diverting the airflow from the tube(s) as part of the complementary flow device 5008. In some forms, the airflow routing may include passages formed in a frame of the mask, and in other forms, in the conduit of a headgear tubing where that tubing forms part of the patient interface. In either form, the passages couple between the tube(s) and the complementary flow device 5008.

Referring to FIG. 8B, the routing 5013, i.e. channels are indicated by dotted-lines. In this Figure, the dotted-lines represent one example of how the routing may be configured, but it is anticipated that other configurations are possible for directing airflow between the tube 5004 and the mask 5006.

Referring to FIG. 8A, similar channels 5012 can be formed in the conduit e.g., of a nasal mask, configured for directing airflow from the tube(s) to the complementary flow device. While a channel, i.e., a lumen, 5012 is indicated (by dotted-lines) on one of the two conduit, it is anticipated that two channels (each located on respective sides of the patient's face) may be provided. In this form, the channels may provide a separate passage for airflow, i.e., being separated from the airflow provided for respiratory therapy.

In either form of the channels 5012, 5013 shown in FIGS. 8A and 8B, the channels may be pneumatically connected to the tube(s), i.e., for receiving air from the flow generator 5002. With reference to FIGS. 8C-1 and 8C-2, it is noted that the channels 5012, 5013 may also be configured to receive air from tubes 5004a or 5004b, and 5005a or 5005b, depending on the type of tubing provided.

It is also noted that, while FIGS. 8C-1 and 8C-2 show two equally sized tubes (i.e., diametrically), one of the tubes may be differently sized. For instance (and as set forth in more detail later) one of the two tubes may be configured, e.g., sized, to connect directly with the channels 5012, 5013.

As set forth in more detail below, the complementary flow device may be configured to control, i.e., regulate, when and how the complementary therapy is administered. For example, when is airflow permitted to pass from the tube(s), through the device 5008; and which of the active or passive therapies are provided.

### 4.8.2 Complementary Flow Device

In either form of the tubing, i.e., single, separate or dual-limbed, the complementary flow device 5008 may be configured to provide either the active or passive therapies. In the case of active therapy, the airflow may be targeted by e.g., a nozzle, so as to apply a stream of pressurised airflow towards the patient, e.g., onto the patient's skin, for stimulating the patient during sleep, i.e. to disturb the patient. Alternatively, or in addition, the secondary airflow may be directed to specific parts of the patient airways. In this form, the secondary air flow delivered from the complementary flow device may be directed within the mask 5006. This may be controlled based on the breath phase. e.g. during inhalation the flow is directed to the nose and/or mouth so the newer, fresher air is inhaled. While during exhalation the flow can be directed to a location(s) of the patient interface to wash out accumulated carbon dioxide. In another form, the secondary air flow may be directed to specific nostrils to be synchronous with the nasal cycles of the patient.

Alternatively, for passive therapy, the airflow may be e.g., diffused through the device 5008 such that the airflow contacts across the patient's skin, i.e. to be distributed across a larger area of the skin than the targeted airflow of active therapy. Advantageously, the diffused airflow may alter the patient's sleeping environment, e.g., by cooling the patient's skin, without disturbing their sleep.

The complementary flow device 5008 may be in the form of a flow regulator valve 5014 for controlling the airflow passing through the device 5008. For example, the valve may be operated to move between opened and closed positions for controlling when airflow moved through the valve. As set forth in more detail later, airflow through the valve may be regulated to control e.g., flow rate, through the device 5008. In this regard, the flow regulator may be used to control whether or not the airflow disturbs the patient's sleep.

In some forms the complementary flow device can be provided with a constant flow vent for delivering a generally constant flow of air towards the patient. The constant flow vent is configured to deliver the generally constant flow of air to the patient regardless of the air pressure output by the flow generator. This may be particularly relevant for e.g., auto-setting devices/flow generators whereby the air pressure output to the patient for complementary therapy may be influenced by the variable airflow of the e.g., auto-setting device. Advantageously, the generally constant flow of air provided by the constant flow vent can provide a more comfortable (i.e., predictable) airflow onto the patient's skin.

As shown in FIGS. 8A and 8B, the complementary flow device may comprise an array of apertures 5008 configured in the mask. The array of apertures (herein referred to as an ‘aperture array’) may be arranged on opposing sides of the mask, i.e., so that left and right sides of the patient may receive the complementary therapy.

Referring to FIG. 8A in particular, the apertures may be arranged with respect to the mask, i.e., to be located in a portion of the conduit headgear, proximal to the cushion module (i.e., 5006).

The location of the apertures, regardless of the type of patient interface, i.e., full-face, nasal pillows, etc., may be positioned according to the anatomical feature to be targeted. For example, in the case of active therapy, the array may be configured to direct air to a typically sensitive area of skin, e.g., proximal the ear(s) (as best shown in FIG. 8B).

In some forms, venting, i.e., vent holes configured to allow washout of exhaled gases, may also be utilised for delivering the complementary therapy. In other words, the apertures 5008 configured for delivering the complementary therapy may also be used to allow washout of exhaled gases such as carbon dioxide.

In some forms, the airflow through the apertures 5008 may be controlled by the patient according to their sensitivity to the airflow. In this form, the apertures may be configured with closures, whereby the patient can adjust the closure to adjust the size, or number of open apertures, so as to control the rate of airflow through the apertures. In some forms, the closures may be arranged to allow the patient to selectively close one or more apertures. For example, the patient may choose to close apertures which direct air to undesirable locations of their face, while leaving some apertures opened to direct air onto more desirable locations.

Advantageously, the closures may allow the patient to customise (e.g. manually adjust) the complementary therapy to suit their sensitivity. For example, some patients may be particularly sensitive to airflow on their skin. In this case, the patient may use the closures to reduce the airflow onto their skin, such that airflow intended to cool the patient does not wake the patient.

The closures may be provided in various forms. For example, the closures may be a slidable plate which the patient can move across the apertures to either open or close the airpath through the apertures. In another example, an adhesive strip can be provided over the apertures for allowing the patient to open and close the apertures by respective peeling away and re-adhering of the adhesive strip over the apertures. In a further example, one or more plugs can be provided for the patient to insert into the apertures, or into a single opening leading to the apertures, for blocking airflow therethrough.

In some forms, the closure can be provided as a textile material configured to cover the apertures 5008 for blocking airflow therethrough. For example, in the form shown in FIG. 8A where the apertures are located on a conduit, a textile sleeve may be provided to overlay the apertures 5008. In this regard, the textile can be configured to either block airflow completely, or be configured with a weave which permits airflow to diffuse therethrough.

In some forms, the textile material can be configured as a type of diffusing material for distributing airflow across the patient's skin. In effect, the textile material may prevent jetting of the airflow onto discrete locations of the patient's skin. This may have a particular application in passive complementary therapy, as set forth in more detail later, whereby the airflow is not intended to disturb/stimulate the user during sleep.

In some forms, the array of apertures 5008 may be located in a discrete location as indicated by reference 5008d shown in FIG. 8A, of a length of the conduit. In alternative forms, the array of apertures 5008 may be spaced, i.e., distributed along the length of the conduit as indicated by reference 5008s. In this form, the apertures may be spread across the length of the routing/channels 5012 so as to direct airflow from the channels. In other forms, the apertures may not be configured with respect to the channels, rather, the apertures 5008 may be configured to direct airflow from the conduit, i.e., for outputting airflow of the conduit that is also used for respiratory therapy. As set forth previously, the apertures in either form may also be configured as venting for washout of exhaled gases.

In forms whereby the apertures 5008s are spread across the channels, the apertures may provide a ‘cooling’ affect for the patient. The apertures may be arranged to face the patient, i.e., to face the skin, or angled to direct airflow to at least partially contact the skin. As set forth above, the apertures and conduit may be covered in a textile sleeve for diffusing the airflow output from the apertures. Advantageously, the direction of airflow towards the patient's skin may provide a feeling of coolness, i.e., a cool temperature, for the patient during use. In effect, this may improve the patient's comfort during respiratory therapy.

In some forms, the airflow may be directed within the seal-forming structure, i.e. to be directed at anatomical features of the patient enclosed by mask. In this form, the airflow (whether active or passive) may be directed to contact either the patient's skin, or be directed towards the patient's airways.

The patient's skin enclosed by the mask may be targeted in the same way as the patient's skin located outside the mask, i.e. with either active or passive airflow.

Advantageously, directing the airflow within the mask, i.e. to be contained within the seal-forming structure, maintains the positive airway pressure formed by the flow generator. As set forth in more detail later, diverting airflow from the mask may introduce additional pressure losses in the system, which require a corresponding increase in power/output from the flow generator to compensate.

In other forms of the complementary flow device, the complementary airflow may be delivered as a controlled leak from the mask seal. For example, the mask seal may be a foam construction that provides a discrete region with intentional/controllable leak through the foam structure/matrix.

In this form, the amount of leak provided by the e.g., foam interface, may be adjusted according to the type of complementary therapy being provided. For example, a high volume of leak through the interface may be used for active therapy, whereby the leak disturbs a patient's sleep. Alternatively, a low volume of leak may be used to cool the patient's skin.

Further, the leaky interface may be constructed to leak within predictable and predetermined limits and physical locations, e.g., vary airflow permeability or breathability proximal to the patient's eyes. In the case of a leaky foam interface, the material of the foam may be selected according to its permeability so as to assist in controlling the magnitude of leak.

Referring now to FIG. 9, the complementary flow device may be configured to deliver both active and passive types of complementary therapy. In the variation shown, a first array of apertures 5008a may be provided, e.g., proximal to the nose bridge and configured to direct (i.e., active) airflow onto the patient's face. A second array of apertures 5008b may be provided on the mask that direct diffused (i.e., passive) airflow onto the patient's face.

The aperture arrays 5008a, 5008b may be in a replaceable component of the mask assembly, e.g., a cushion component. Advantageously, this arranges the apertures proximal to the patient's skin, so as to ensure airflow is directly contacting e.g., the skin, rather than inadvertently contacting e.g., the patient's eyes. Further, providing the apertures on the replaceable component allows the location of the holes to be adjusted depending on a design of the replaceable component.

For example, in the case of a cushion, the location and arrangement of the apertures may be changed for each of the available sizes of cushion. That is, a large cushion may have the apertures located lower with respect to the nose bridge, when compared to a small sized cushion. In effect, this can ensure the airflow from the apertures contacts generally the same region of the patient's face.

Further, providing the apertures on the replaceable component may allow the patient to either use a cushion with the apertures 5008 (for complementary therapy) or to use a cushion without the apertures (i.e., electing not to have complementary therapy). In other words, some patients may like to use complementary therapy, and so can optionally apply a cushion providing apertures to their mask assembly.

Each of the aperture arrays 5008a, 5008b (i.e., for either passive or active therapy) may be connected to the tubing 5004 via routing (best shown in FIG. 8B), i.e., passages formed in the mask. In this form, the complementary flow device 5008 may be configured as a type of ‘passive/active switch’ that is able to control whether airflow passes through the active or passive aperture array. For example, if the patient is required to be cooled, the device 5008 may be controlled to direct airflow through the passive aperture array (5008b). If, at a later time, the patient is required to be woken or disturbed (e.g., for positional therapy), the device 5008 may be controlled to direct airflow through the active aperture array (5008a).

As set forth above, the device 5008 in the form of the ‘passive/active switch’ may also be utilised to regulate e.g., pressure, velocity, etc., of the airflow passing through either of the aperture arrays 5008a,5008b. For example, the device may be partially opened limit passage of airflow therethrough.

### 4.8.3 Flow Regulator

The system 5000 may comprise a flow regulator valve 5014 (herein, the ‘flow regulator 5014’) for controlling at least a part of the airflow passing through the apparatus 5000. The flow regulator 5014 can be utilised to control a passage of airflow through aperture arrays 5000c, as well as aperture arrays 5008a, 5008b, 5008d, 5008s (i.e., for either passive or active therapy) via the channels/routing 5012, 5013 (as set forth previously in relation to see FIGS. 8A and 8B).

Referring to the embodiments shown in FIGS. 10 to 15, the flow regulator 5014 can be configured to divert a proportion of the flow of respiratory gas flowing into the routing 5012, 5013 from headgear tubes 2350 (as per FIG. 8A, for pillow mask) or tube 5004 (see FIG. 8B, for e.g. full-face masks).

The flow regulator 5014 can comprise a body 5016 having an inlet 5018, an aperture array 5008c, a barrier arrangement 5024, and first and second outlets 5020a,5020b corresponding with respective first and second headgear tubes 2350.

The flow regulator 5014 may form a junction to connect between the tube 5004 and the headgear tubes 2350. In other forms (not shown), the flow regulator 5014 may form a junction between the headgear tubes 2350 and aperture arrays of the device as set forth previously, e.g., aperture arrays 5000a and 5008b (i.e., in the cushion), 5008d and 5008s (i.e., along a length of the tube). In some further forms again, the flow regulator 5014 may form a junction between the tube 5004 and a mask, i.e., as best shown in FIG. 8B connecting between the tube and channels 5013 of the mask.

Referring to a first form of the first embodiment as shown in FIG. 10A, the apertures 5008c may be formed in a base 5022 of the body 5016. In the form shown, the flow regulator is contained within the headgear tube, i.e., conduit. In this form, the apertures 5008c also extend through the headgear tube.

As set forth previously, the tube 5004 can convey the flow of respiratory gas to the headgear tubes or masks (e.g., a full-face mask). While the apertures can divert a portion of the respiratory gas onto the patient, the flow regulator 5014 is configured to convey a majority of the flow of respiratory gas into, e.g., the nasal cushion, full-face mask, etc, for delivering an appropriate CPAP therapy.

As shown in FIG. 10A, when viewed from a front of the apparatus, the apertures 5008c are generally orientated centrally of the base 5022, so as to substantially align with a central region of the patient's head, in-use. The flow regulator 5014 may be arranged with respect to the routing 5012,5013 to connect between other apertures to control/regulate flow therethrough, e.g. at aperture arrays 5000a, 5008b, 5008d, 5008s.

In the first embodiment, the barrier arrangement 5024 is in the form of a wedge-shaped pendulum (herein referred to as the ‘pendulum 5024’). The pendulum 5024 may be located in a chamber 5026 defined by the body 5016 and may be suspended at its apex 5028 such that the pendulum 5024 can pivotable within the chamber (see FIGS. 10B and 10C).

In this embodiment, the pendulum 5024 is a gravity actuated component whereby the pendulum 5024 moveable under gravitational forces between a first configuration, shown in FIG. 10B, and a second configuration, shown in FIG. 10C.

As best shown in the side views of FIGS. 10B and 10C, the apertures 5008c are located towards one side of the body 5016, i.e., offset from a central region thereof. The apertures 5008c are located such that, in-use, when the positioning and stabilising structure is worn by the patient, the apertures are located towards the patient's forehead, e.g., towards their frontal bone. In some forms (not shown), the flow regulator 5014 can be configured such that the apertures are located towards a rear of the patient's head, e.g., towards their parietal bone. In this position, the apertures 5008c are aligned so that a portion of the flow of respiratory gas is directed towards the patient.

The pendulum 5024 is configured such that, when the patient is lying on their back, i.e., when their head is not tilted towards one side, the pendulum 5024 moves, i.e., swings, away from the apertures. This position of the pendulum, i.e., a resting position, is shown in FIG. 10B, and allows airflow to pass through the apertures so as to contact, i.e., blow onto, the patient.

When the apertures 5008c are opened, the respiratory gas can flow therethrough, ‘jetting’ air onto the patient at a high speed. In some forms of the flow regulator 5014, this jetting may be utilised to disturb the patient from their sleep, as a result of the jetting being uncomfortable for a patient. In this form, the apertures may be positioned to directly contact the patient's skin. Advantageously, configuring the apertures to jet air onto e.g., the patient's forehead, when they are laying on their back can encourage them to move away from the back sleeping position, i.e., into a side sleeping position.

In some alternative forms of the flow regulator 5014, the apertures may be arranged to direct airflow onto a patients' hair. Advantageously, the patient's hair may act as a diffuser to diffuse the jetting action of the airflow. This can create a more ‘comfortable’, i.e., less disturbing flow of air onto the patient.

In some further forms again, a diffuser may be utilised together with the flow regulator 5014. The diffuser may be a mesh-like material located with respect to the aperture such that airflow therethrough is diffused. This can ‘soften’ the pressure of the airflow contacting the patient. Advantageously, this may be used to gently awaken a patient, or alternatively, cool a patient during sleep in order to improve their sleep.

The orientation of the patient's head may be determined by the side to which the patient's head is tilted towards. If the patient's head is tilted slightly or substantially towards their left or right side, the pendulum 5024 is configured to pivot towards the apertures 5008c. This position of the pendulum, i.e., a closed position, is shown in FIG. 10C. The pendulum in this position, i.e., a forward position, blocks the passage of air flow through the apertures 5008c so that air flow no longer contacts the patient.

The wedge-shape of the pendulum provides an offset mass that, advantageously, biases the pendulum into either the resting position (i.e., whereby the apertures are open) or the closed position (i.e., where the apertures are closed). In this regard, the pendulum can be considered ‘bi-stable’, i.e., having more than one location at which it may be stable. The bi-stable characteristic means that the pendulum does not have one ‘stable’ position that it moves towards/into.

Biasing the pendulum 5024 into either the resting position or the closed position can ensure the pendulum 5024 does not move away from either of the resting or closed positions until the patient has completely changed sleeping positions, e.g., moved from a back sleeping orientation to a side sleeping orientation. In other words, the position of the pendulum can be dependent on the sleeping position of the patient.

Advantageously, the offset mass of the wedge-shape can stabilise the pendulum 5024 during ‘slight’ movements of the patient during their sleep. Configuring the barrier arrangement 5024 in this way can also ensure that airflow is only allowed to pass through the apertures once the pendulum 5024 is positioned away from the closed position i.e., disturbing the patient only when they have moved into a back sleeping orientation.

Referring now to FIGS. 11 to 13, a second form of the first embodiment is shown. In this form, the pendulum 5024 can comprise an array of corresponding apertures 5030 therein for allowing a passage of airflow therethrough. The pendulum 5024 in this form is movable, i.e., pivotable, from a central configuration (as shown in FIGS. 11 and 12A) to a first configuration (as shown in FIG. 12B) and a second configuration (as shown in FIG. 12C).

This second form of the pendulum primarily differs from the first form in that the second form pivots from ‘side-to-side’ of the in-use device, whereas the first form pivots from ‘front-to-back’.

As shown in FIGS. 11 and 12A, when the pendulum 5024 is in the central configuration, i.e., positioned substantially in a central region of the chamber 5026, the apertures 5030 of the pendulum 5024 align with the apertures 5008c of the body 5016. This alignment of apertures 5030,5008c allows a passage of respiratory air (from the inlet 5018) to flow through the apertures 5030,5008c to contact the patient.

Referring specifically to FIG. 12B, the pendulum 5024 is configured to be moved by gravity towards the patient's first side when the patient's head is oriented towards the first side. Conversely, as shown in FIG. 12C, the pendulum 5024 is configured to be moved by gravity towards the patient's second side when the patient's head is oriented towards the second side.

Referring to FIGS. 11 and 12A, in use, the pendulum 5024 is configured to be located in the central configuration, i.e., central region of the chamber 5026 when the patient's head is not tilted towards the first side or the second side e.g., when they are lying on their back.

In this position, the apertures 5008c,5030 of the base 5022 and the pendulum 5024 are aligned so that a flow of respiratory gas is directed towards the patient.

Referring now to FIGS. 12B and 12C. When the patient's head is oriented towards one side, the pendulum 5024 can pivot to the first or second configurations so that the apertures 5008c,5030 are no longer aligned. In effect, the pendulum 5024 blocks the passage of air flow through the apertures 5008c,5030.

In this second form, the extent of displacement of the pendulum 5024 from its central configuration can be dependent on the angle at which the patient's head is oriented towards either the first side or the second side. Accordingly, the extent to which the respective apertures 5030,5008c of each component align can affect the magnitude of air flow therethrough. That is, the proportion/magnitude of the flow of respiratory gas through the apertures (and into contact with the patient) can be based on the orientation of the patient's head.

For example, when a patient is sleeping on their back, a slight movement of the patient's head to either side may result in a partial closing of the apertures 5008c. Advantageously, this can provide a ‘positive reinforcement’ for a patient, promoting movement of their body into a side sleeping orientation.

Referring now to FIG. 13, in some forms of the apparatus 5000, the flow regulator 5014 may form a junction to connect the tube 5004 and routing 5012,5013 for connecting to the aperture arrays 5000a, 5008b, 5008d, 5008s. In this form, the flow regulator 5014, including the pendulum 5024 and apertures 5030, can otherwise be configured as set forth previously, i.e., whereby the pendulum does not include apertures therein.

In this form, the routing may extend through the base 5022 of the flow regulator 5014 and through e.g., a frame of the mask (as per FIG. 8B) or through the conduit of headgear tubing 2350 (as set forth previously). In this regard, when the pendulum 5024 is located in the resting position or in the central configuration, i.e., when the patient is sleeping on their back, the routing 5012,5013 may be opened so that a flow of respiratory gas is directed through the routing to aperture arrays 5000a, 5008b, 5008d, 5008s. When the patient's head is oriented towards one side, the pendulum 5024 can pivot to the closed position (as per FIG. 10C) or into the first or second configurations (as per FIG. 12B or 12C), so that the routing passages 5012,5013 are no longer open. These positions of the pendulum thereby block the passage of air flow therethrough.

Referring now to FIGS. 14A to 14C and FIGS. 15A to 15C, a second embodiment of the flow regulator 6014 is shown. In the following description of the second embodiment, like reference numerals are used for like features with the addition of the prefix ‘6’.

The second embodiment of the flow regulator 6014 primarily differs from the first embodiment of the flow regulator 5014 in that the barrier arrangement 5024 is configured as a slider 5024. The slider 5024 can have a rectangular or square cross-sectional area and can be configured for sliding between first and second configurations, as set forth in detail below.

In a first form of the second embodiment, as shown in FIG. 14A-14C, the slider 6024 can comprise apertures 6030 configured to align with apertures 6008c in a base 6022 of the flow regulator when the slider 6024 is in a central configuration. In the central configuration, the apertures 6030 of the slider align with the apertures 6008c of the base to permit a passage of air flow therethrough.

The base 5022 can comprise stops 6032 between which the slider 6024 can move. As shown in FIGS. 14B and 14C, the slider 6024 can be configured to freely slide between the stops 6032. In this regard, the slider may be weighted so that it may be a gravity actuated, i.e., slidable, component.

In-use, when the patient's head is oriented towards one side, the slider 6024 can move away from the central configuration and into either of the first or second configurations. In the first or second configurations, the apertures 6030 are no longer aligned with the apertures 6008c, thereby blocking the passage of air flow therethrough.

In a second form, as shown in FIG. 15A-15C, the slider 6024 can comprise apertures 6030 configured to align with routing 5012,5013 in the base 6022 when the slider 6024 is in the central configuration.

As shown in FIGS. 15B and 15C, when the flow regulator 6014 is oriented towards one side, the slider 6024 can slide away from the central configuration and into either of the first or second configuration. In each of these configurations, the routing 5012,5013 is closed such that a passage of air flow therethrough is blocked.

As set forth in more detail later, the flow regulator 6014 may comprise sensors, actuators and processing systems to determine when the air flow should be diverted to the apertures for contacting a patient. In this form, the actuators may be configured to control a position of the slider, operating on a power supply rather than gravity.

### 4.8.4 Sensory Monitoring and Stimulation Unit

The sensory monitoring and stimulation unit 5010 may be configured to determine when the passive and active therapies are operated. That is, the unit 5010 may be connected to one or more sensors for measuring parameters of the ambient environment or health metrics of the patient. The sensors may be located on, or proximal to the mask 5006 or flow generator 5002. In some forms, the sensors may be alternatively or additionally located on a portion of the tubing, 5004.

The sensors used for detecting the patient's health metrics, i.e., physical condition, may measure e.g., heart rate, perspiration, temperature, breath rate, oxygen-saturation, etc. Accordingly, the sensors utilised to detect such parameters may include a heart rate sensor, moisture sensor, thermistor, flow sensor, oximeter, etc.

The sensors configured to detect the ambient environment around the patient may measure e.g., ambient air temperature, humidity, pressure, etc. The information read by the sensors (either for health metrics or ambient environment) may be logged in real-time (e.g., in the memory of the RPT device) and may be later recalled to pre-emptively adapt e.g., the sleeping position or the sleeping environment of a patient.

The sensory monitoring and stimulation unit 5010 may be integrated, i.e., connected with a controller (not shown) of the apparatus 5000. The controller may be configured to control the operation of the flow generator 5002 based on input from the sensory monitoring and stimulation unit. The controller may be used, for example, to adjust the flow rate of the flow generator, to adjust the air pressure generated by the flow generator, etc.

In some forms, the complementary flow device 5008, e.g., when configured as a flow valve, may be regulated by input from the sensory monitoring and stimulation unit 5010 for automatically adjusting e.g., the flowrate during use. In this regard, the type of airflow directed onto the patient (i.e., active or passive) may be adjusted according to changes occurring during sleep (i.e., of the patient or of the environmental).

The sensory monitoring and stimulation unit 5010 may also be configured to determine, e.g., whether a patient is e.g., sleeping on their back and thereby activate either the left or right side of the aperture array to e.g., cool or alert the patient to change sleeping position. Advantageously, this selective activation of the aperture arrays may accommodate for when the apertures may be occluded when e.g., a pillow or other object covers the array. Further, this may be particularly advantageous for active therapy, as set forth in more detail later, whereby for e.g., positional therapy, airflow is utilised to stimulate the patient to e.g., roll from a back-sleeping position to sleeping on their side.

In some forms, the controller may be housed in the flow generator 5002 and coupled to the visual display, e.g., a Liquid Crystal Display (LCD) of the flow generator. The display may allow for a patient (or other user) to optionally adjust settings of the complementary therapy, e.g., on/off, in-use airflow rate, airflow pressure, airflow temperature, and airflow humidity, etc. Alternatively, the settings of the complementary therapy may be operated by a control device (not shown). The control device may be a smartphone, computer, standalone device, etc. In some forms, the control device may be configured to wirelessly communicate with the controller to allow for a patient (or another user) to remotely control operation of the complementary therapy.

### 4.8.5 Passive Complementary Therapy

As set forth above, passive complementary therapy may be used to improve the sleeping environment of a patient. In some cases, changes in the patient's sleeping environment such as temperature, lighting, noise, etc, may correlate with interruptions in the patient's sleep. Ultimately, this passive complementary therapy aims to improve a patient's sleep health by positively influencing factors such as sleep latency, sleep waking, wakefulness, sleep efficiency, etc.

Passive complementary airflow may be used to positively affect these factors by creating changes in the patient's sleeping environment without disturbing their sleep. As set forth previously, in some forms, passive airflow may be directed to a patient's face to cool them if their temperature is elevated during sleep.

In this form, the airflow may be provided at ambient room temperature, whereby the movement of air across the patient's face is sufficient to lower their body temperature. In other forms, the airflow delivered from the flow generator 5002 may be cooled to more effectively lower the patient's temperature. For example, the airflow may be cooled by use of a Peltier chip.

It should also be noted that, while the airflow may be used for cooling a patient, the airflow may be modified in other ways so as to improve a patient's sleep. For instance, in cases where the patient's body temperature is lowered, the airflow may be warmed so as to warm the patient. In this regard, the heating element of the humidifier or tubing 5004 (or in some cases, utilising airflow from the plenum chamber) may be utilised to warm airflow delivered for the passive therapy. In this regard, the temperature of the air may also be utilised for active therapy, i.e., to stimulate the patient from sleep. For example, cool air may be delivered to more effectively alert the patient, i.e., rather than utilising ambient (i.e., warmer) temperature air.

In some forms, the airflow directed e.g., into the channels may be configured to adapt the headgear to fit the patient's face. For example, in the case of a conduit headgear, the airflow directed into the channels 5012 may be configured to change the shape and/or size of the channels 5012 so as to also change the shape and/or size of the headgear to more appropriately fit, i.e., conform, to the patient's face. In this case, the flow generator may be configured with an algorithm to detect e.g., unintentional leak from the mask, and thereby use the airflow directed into the channels 5012 to change the shape of the headgear so as to e.g., pull the mask 5006 tighter (into sealing contact) against the patient's face.

In another example, the airflow directed into the channels 5012 may be configured to distribute loads applied by the headgear to e.g., a bony portion, of the patient's face. In this case, the airflow may adapt, e.g., deform, the headgear such that the headgear may have a wider contact area with the patient's skin. This may be particularly relevant for the conduit headgear, but may also be utilised for headgear not comprising a conduit.

Referring now to other uses of the passive airflow, the passive airflow may positively impact a patient's sleep health by influencing a duration of sleep. That is, the passive complementary therapy may be used to wake a patient once an appropriate amount of sleep has been achieved. In this case, rather than provide an airflow that does not disturb the patient's sleep, the sensory monitoring and stimulation unit 5010 may be configured to apply a stimulating, i.e., more aggressive, type of airflow to wake the patient from sleep. In this regard, the airflow may be used as a type of ‘alarm clock’, the stimulating airflow may be intended to wake a patient by directing airflow to a sensitive part of the patient's face.

Notably, the use of airflow to wake a patient may be done so according to the patient's sleep state. For example, the sensory monitoring and stimulation unit 5010 may be utilised to detect the sleep state of a patient, then direct the controller to supply the airflow at a particular time-point during the sleep cycle in order to wake the patient at an ideal/optimal point in time, e.g., during light sleep. Advantageously, determining the ideal time to alert the patient may avoid disturbing beneficial sleep states, e.g., deep sleep, REM sleep, etc.

The sensory monitoring and stimulation unit 5010 may be configured to automatically adjust how the complementary therapy/airflow is delivered based on information received from the sensors. It is also anticipated that the sensory monitoring and stimulation unit may be configured to collect information from an online resource, e.g., a weather (current or forecast), to assess the ambient environment in which the patient will sleep. For example, air quality (e.g., pollen levels), temperature, humidity, etc. In turn, the sensory monitoring and stimulation unit may adjust e.g., the type, magnitude, etc, of the complementary therapy provided to the patient for the duration of their sleep.

Information received by the sensors may be utilized to cause the system to automatically adjust certain parameters of the complementary therapy. For example, measurements of the ambient air temperature taken during a patient's sleep may cause the flow device 5008 and/or the flow generator 5002 to modify operation in real-time e.g., by adjusting the magnitude, temperature, etc., of the airflow.

In some forms, the sensory monitoring and stimulation unit 5010 may be configured to prompt the patient to manually adjust the delivery of the complementary therapy. For example, if the body temperature of the patient is detected to be increasing, the unit 5010 may be configured to alert the user, e.g., by audio alarm, visual prompt on the device display, etc, to adjust the complementary therapy to e.g., direct cool air onto the patient.

The sensory monitoring and stimulation unit 5010 may also be configured to receive information from the sensors and/or from online resources to provide feedback to the patient about their sleep. For example, information about the patient's sleep environment may be provided e.g., through a smart phone or other internet connected device. Such feedback may inform the patient as to e.g., why their sleep may have been disturbed/interrupted.

### 4.8.6 Active Complementary Therapy

As set forth previously, active complementary therapy may be used for treating respiratory disorders such as OSA. As previously exemplified, in some forms the active airflow may be used for positional therapy, to stimulate movement of the patient during an OSA event. In this case, the complementary airflow may be configured for stimulating the patient to alert them to change their sleeping position, i.e., to move into a side sleeping position.

The sensory monitoring and stimulation unit 5010 may be configured to sense a patient's sleeping position and, based on the sleeping position detected, utilise airflow through the complementary flow device 5008 to stimulate the patient to move from their sleeping position. This may be particularly useful for patients who are more likely to experience sleep apnoea when e.g., sleeping on their back. For example, in such a scenario where the patient is detected as sleeping on their back, the unit 5010 can deliver airflow to stimulate the patient. The airflow is intended to prompt, i.e., initiate the patient to move to e.g., a side-sleeping position so as to reduce the chances of experiencing a sleep apnoea event.

Advantageously, moving the patient off their back during sleep means that the flow generator can operate at a lower pressure, i.e., since a patient's airway may naturally open when the patient is laying on their back. In effect, the lower operating pressure means the system is generally quieter (compared to when operating at higher pressures), thus, the patient is less disturbed by e.g. noise, and thereby may be more comfortable.

In some forms, the airflow delivered through the complementary flow device 5008 can contact the patient's skin in order to stimulate the patient. The complementary flow device may also be configured to direct air onto a patient's face, for example, towards the patient's eyes, cheeks, chin, etc.

The location to which the airflow is directed and the pressure, velocity, etc of the airflow may each be adjusted according to the magnitude of stimulation required. For example, different regions of the patient's face may be more or less sensitive to airflow, and so, airflow may be directed to specific locations according to the type of response required from the patient.

### 4.8.7 Flow Generator

It is anticipated that respiratory pressure therapies, e.g., CPAP therapy, typically require higher airflow rates from the flow generator than those provided for the passive or active complementary therapy. As such, a flow generator configured for use with both respiratory and complementary therapy may be required to generate a higher output pressure than for use with respiratory pressure alone.

For example, the flow generator will be required to overcome additional pressure losses within the system. These may include losses as the airflow travels through one or more of the tubes, routing within the mask/conduit, venting, complementary flow devices, and connectors therebetween. The magnitude of losses may also vary according to a number of other parameters, including the geometry of the pneumatic path etc.

Accordingly, the flow generator may be configured, e.g., with a motor, power output, etc., that can maintain the required therapeutic pressure for e.g. CPAP while additionally providing a source of complementary airflow.

Further, the flow generator can be configured to identify the complementary airflow as an ‘intentional leak’, being different from unintentional leak. Advantageously, configuring the flow generator in this way means that the airflow from the complementary therapy is not detected/recorded as unintentional leak, allowing the flow generator to operate with the appropriate functions, e.g. pressure, etc.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

4.9.1.1 Materials & their Properties

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.9.1.2 Mechanics

### Axes:

a. Circumferential axis: An axis oriented perpendicularly with respect to the longitudinal axis. The axis may be specifically present in pipes, tubes, cylinders, or similar shapes with a circular and/or elliptical cross section.

Elasticity: The ability of a material to return to its original geometry after deformation.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

### 4.9.1.3 Structural Elements

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Tie (noun): A structure designed to resist tension.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 4.9.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Tᵢ to total breath time, Tₜₒₜ.

Effort (breathing): The work done by a spontaneously breathing person attempting to breathe.

Expiratory portion of a breathing cycle: The period from the start of expiratory flow to the start of inspiratory flow.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Tᵢ): The duration of the inspiratory portion of the respiratory flow rate waveform.

Exhalation Time (Tₑ): The duration of the expiratory portion of the respiratory flow rate waveform.

Total Time (Tₜₒₜ): The total duration between the start of one inspiratory portion of a respiratory flow rate waveform and the start of the following inspiratory portion of the respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.9.3 Ventilation

Expiratory positive airway pressure (EPAP): a base pressure, to which a pressure varying within the breath is added to produce the desired interface pressure which the ventilator will attempt to achieve at a given time.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP-EPAP). In some contexts, pressure support means the difference which the ventilator aims to achieve, rather than what it actually achieves.

### 4.9.4 Anatomy

### 4.9.4.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 4.9.4.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.9.4.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.9.5 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.9.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the case of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.11 Reference Signs List

| | |
|---|---|
| patient | 1000 |
| bed partner | 1100 |
| patient interface | 3000 |
| seal-forming structure | 2100 |
| cushion module | 2150 |
| plenum chamber | 2200 |
| chord | 2210 |
| superior point | 2220 |
| inferior point | 2230 |
| plenum chamber inlet port | 2254 |
| positioning and stabilising structure | 2300 |
| headgear | 2302 |
| four-point headgear | 2302-1 |
| two-point headgear | 2302-2 |
| strap | 2310 |
| inlet | 2332 |
| tube | 2350 |
| vent | 2400 |
| groove | 2416 |
| connection port | 2600 |
| elbow | 2610 |
| forehead support | 2700 |
| RPT device | 3000 |
| external housing | 3010 |
| upper portion | 3012 |
| portion | 3014 |
| panel | 3015 |
| chassis | 3016 |
| handle | 3018 |
| pneumatic block | 3020 |
| air filter | 3110 |
| inlet air filter | 3112 |
| outlet air filter | 3114 |
| muffler | 3120 |
| inlet muffler | 3122 |
| outlet muffler | 3124 |
| pressure generator | 3140 |
| blower | 3142 |
| motor | 3144 |
| anti-spill back valve | 3160 |
| air circuit | 3170 |
| air circuit | 3171 |
| supplementary gas | 3180 |
| electrical components | 3200 |
| single Printed Circuit Board Assembly PCBA | 3202 |
| power supply | 3210 |
| input device | 3220 |
| central controller | 3230 |
| clock | 3232 |
| therapy device controller | 3240 |
| protection circuits | 3250 |
| memory | 3260 |
| transducer | 3270 |
| pressure sensor | 3272 |
| flow rate sensor | 3274 |
| motor speed transducer | 3276 |
| data communication interface | 3280 |
| remote external communication network | 3282 |
| local external communication network | 3284 |
| remote external device | 3286 |
| local external device | 3288 |
| output device | 3290 |
| display driver | 3292 |
| display | 3294 |
| humidifier | 4000 |
| humidifier inlet | 4002 |
| humidifier outlet | 4004 |
| humidifier base | 4006 |
| reservoir | 4110 |
| conductive portion | 4120 |
| humidifier reservoir dock | 4130 |
| locking lever | 4135 |
| water level indicator | 4150 |
| heating element | 4240 |
| apparatus for respiratory therapy and | 5000 |
| complementary therapy | |
| flow generator | 5002 |
| air conduit/tube | 5004 |
| tube branch | 5004a |
| tube branch | 5004b |
| separate tube | 5005a |
| separate tube | 5005b |
| patient interface/mask | 5006 |
| complementary flow device | 5008 |
| apertures configured for active airflow | 5008a |
| apertures configured for passive airflow | 5008b |
| sensory monitoring and stimulation unit | 5010 |

1. An apparatus for delivery of pressurised air or breathable gas to a patient, the apparatus comprising:
a flow generator configured to generate a flow of air;
a patient interface constructed and arranged to form a seal with a region of the patient's face surrounding an entrance to the patient's airways, the patient interface being configured to deliver the pressurised air or breathable gas to the patient's airways for respiratory therapy; and
an air delivery tube coupled between the flow generator and the patient interface to deliver a first portion of the flow of air from the flow generator to the patient interface as the pressurised air or breathable gas; and
a complementary flow device configured to deliver a second portion of the flow of air to the patient as a complementary activity to the respiratory therapy.

2. An apparatus according to claim 1, wherein the second portion of the flow of air is delivered away from the patient's airways.

3. An apparatus according to claim 2, wherein the complementary flow device is configured to deliver the second portion of the flow of air outside the patient interface.

4. An apparatus according to claim 2, wherein the complementary flow device is configured to deliver the second portion of the flow of air onto the patient's skin.

5. An apparatus according to claim 4, wherein the complementary flow device is configured to target a discrete location of the patient's skin with the second portion of the flow of air to stimulate a response in the patient that forms at least part of an active complementary therapy.

6. An apparatus according to claim 4, wherein the complementary flow device is configured to diffuse the second portion of the flow of air across the patient's skin to alter the environment around the patient.

7. An apparatus according to claim 1, wherein the second portion of the flow of air is directed to one or more specific areas of the patient airways as the complementary activity.

8. An apparatus according to claim 1, wherein the patient interface comprises a frame configured to conform to the shape of the patient's face, the complementary flow device forming part of the patient interface and being arranged in the frame to direct the second portion of the flow of air as part of the complementary therapy.

9. An apparatus according to claim 8, wherein the complementary flow device comprises routing to direct the second portion of the flow of air, the routing provided at least in part on the frame of the patient interface.

10. An apparatus according to claim 1, comprising more than one complementary flow device for directing the second portion of the flow of air towards more than one location the more than one complementary flow device are for delivering more than one of active complementary therapy to the patient where the diverted airflow is configured to stimulate a response in the patient and/or passive complementary therapy where the diverted airflow alters the environment around the patient.

11. An apparatus according to claim 1, wherein the complementary flow device comprises an array of apertures configured to direct the flow of air.

12. An apparatus according to claim 1, the apparatus further comprising a flow regulator valve for regulating the second portion of the flow of air to the patient, the flow regulator configured to move between an open state for allowing airflow therethrough, and a closed state for blocking airflow therethrough, wherein the state of the flow regulator valve between the open and closed states is dependent on an orientation of the apparatus.

13. An apparatus according to claim 12, wherein the flow regulator valve is arranged to move between the closed and open states under gravity.

14. A patient interface for delivery of pressurised air or breathable gas to a patient, the patient interface constructed and arranged to form a seal with a region of the patient's face surrounding an entrance to the patient's airways to deliver the pressurised air or breathable gas to the patient's airways for respiratory therapy, the patient interface further comprising a complementary flow device configured to divert at least part of the pressurised air or breathable gas as a complementary activity to the respiratory therapy.

15. A patient interface according to claim 14, wherein the patient interface comprises a frame configured to conform to the shape of the patient's face, the complementary flow device forming part of the patient interface and being arranged in the frame to direct the diverted flow of air as part of the complementary activity.

16. A patient interface according to claim 15, wherein the complementary flow device comprises routing to direct the diverted flow of air, the routing provided at least in part on the frame of the patient interface.

17. A patient interface according to claim 14, further comprising a flow regulator valve for regulating the second portion of the flow of air to the patient, the flow regulator configured to move between an open state for allowing airflow therethrough, and a closed state for blocking airflow therethrough, wherein the state of the flow regulator valve between the open and closed states is dependent on an orientation of the patient interface.

18. A system for delivery of pressurised air or breathable gas to a patient, the system comprising: an apparatus for delivery of pressurised air or breathable gas to a patient, the apparatus comprising:
a flow generator configured to generate a flow of air;
a patient interface constructed and arranged to form a seal with a region of the patient's face surrounding an entrance to the patient's airways, the patient interface being configured to deliver the pressurised air or breathable gas to the patient's airways for respiratory therapy; and
an air delivery tube coupled between the flow generator and the patient interface to deliver a first portion of the flow of air from the flow generator to the patient interface as the pressurised air or breathable gas;
a complementary flow device configured to deliver a second portion of the flow of air to the patient as a complementary activity to the respiratory therapy; and
a sensory monitoring and stimulation unit and a controller configured with respect to the flow generator to set an operation of the complementary flow device.

19. A system according to claim 18, wherein the sensory monitoring and stimulation unit is coupled with one or more sensors configured to detect physiological data of the patient, the controller configured to set the operation of the complementary flow device based on a signal from the one or more sensors.

20. A system according to claim 18, wherein the sensory monitoring and stimulation unit is coupled with one or more sensors configured to detect data of the patient's sleep environment, the controller configured to set the operation of the complementary flow device based on a signal from the one or more sensors.

21. A method for delivering pressurised air or breathable gas to a patient comprising:
arranging a patient interface to form a seal with a region of the patient's face surrounding an entrance to the patient's airways;
generating a flow of air to be delivered to the patient interface as the pressurised air or breathable gas;
delivering a first portion of the flow of air as the pressurised air or breathable gas to the patient's airways via the patient interface for respiratory therapy; and
delivering a second portion of the flow of air to the patient as a complementary activity to the respiratory therapy.

22. A method according to claim 21, wherein at least part of the second portion of the flow of air is directed away from the patient's airways.

23. A flow regulator valve for use with an apparatus for delivery of pressurised air or breathable gas to a patient for respiratory therapy, the flow regulator valve configured for regulating a flow of air to the patient as a complementary activity to the respiratory therapy, the flow regulator configured to move between an open state for allowing airflow therethrough, and a closed state for blocking airflow therethrough, wherein the state of the flow regulator valve between the open and closed states is dependent on an orientation of the apparatus.

24. A flow regulator valve according to claim 23, wherein the flow regulator valve is arranged to move between the closed and open states under gravity.
